(19) United States
(12) Patent Application Publication
(10) Pub. No.: US 2024/0103020 A1
(43) Pub. Date: Mar. 28, 2024

(54) MATERIALS AND METHODS FOR DIAGNOSIS

(71) Applicant: QSM Diagnostics, Inc., Boston, MA (US)

(72) Inventors: Edgar D. GOLUCH, Somerville, MA (US); Nicholas CADIROV, Somerville, MA (US)

(21) Appl. No.: 18/272,428

(22) PCT Filed: Jan. 13, 2022
(86) PCT No.: PCT/US22/12244

§ 371 Date: Jul. 14, 2023

Related U.S. Application Data
(60) Provisional application No. 63/137,882, filed on Jan. 15, 2021.

(51) Int. Cl. G01N 33/68; G01N 27/327; G01N 33/487; G01N 33/49; G01N 33/493

(52) U.S. Cl. CPC G01N 33/6893; G01N 27/3277; G01N 33/48714; G01N 33/492; G01N 33/493; G01N 2800/348

(57) ABSTRACT

The present disclosure relates to, inter alia, materials, and methods for detection of infection. More particularly materials, and methods for detecting an infection in a subject's urine or wound exudate are described, e.g. by electrochemically measuring a target molecule and/or a metabolic activity associated with infection using an electrochemical sensor array.

### PRIORITY

This application claims the benefit of, and priority to, U.S. Provisional Application No. 63/137,882, filed Jan. 15, 2021, the contents of which are hereby incorporated by reference in their entirety.

### TECHNICAL FIELD

The present invention relates to, inter alia, materials, methods and kits for detection of pathogens.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: QSM-002PC_Sequence-Listing_ST25; date created: Jan. 11, 2022; file size: (2,298 bytes).

### BACKGROUND

Urinary tract infections (UTIs) are one of the most common health care acquired infections in the US, with an estimated 93,000 UTIs in acute care hospitals in 2011. See Dudeck et al., National Healthcare Safety Network (NHSN) Report, Data Summary for 2011, Device-associated Module, Am J Infect Control. 41(4): 286-300 (2013). Up to 70-80% of these infections can be attributed to the use of an indwelling urinary catheter. Magill et al., Multistate Point-Prevalence Survey of Health Care-Associated Infections, N Engl J Med. March 27; 370(13): 1198-1208 (2014). Complications from untreated catheter associated urinary tract infections (CAUTI) include more serious bladder and kidney infections, which can lead to bacteremia and sepsis. There are over 4 million patients at long term care (LTC) facilities in the U.S. (Long Term Care CDC 2019), many of which have asymptomatic bacteriuria (estimated at 18% to 57% for women and 19% to 38% for men (Nicolle, Urinary tract infections in long-term-care facilities. Infect Control Hosp Epidemiol. 22(3):167-75 (2001)) that should be monitored for signs of disease progression (Genao and Buhr, Urinary Tract Infections in Older Adults Residing in Long-Term Care Facilities, Ann Longterm Care 20(4): 33-38. (2012)). Many of these infections and complications could be prevented with appropriate monitoring of the catheter and urine collected (Lo, et al., Strategies to prevent catheter-associated urinary tract infections in acute care hospitals: 2014 update, Infect Control Hosp Epidemiol 35(5) 464-79 (2014)). However, there is currently no simple way to quickly determine or monitor for a catheter associated urinary tract infection or impending catheter blockage.

More than 1.25 million, and 6.5 million people suffer from burns and chronic skin ulcers, respectively, every year in the United States. Chronic skin ulcers are caused by pressure, venous stasis, or diabetes mellitus. Singer and Clark, Cutaneous wound healing. N Engl J Med. 341(10):738-46 (1999). Chronic wounds are increasing with the surge in prevalence diabetes and obesity. Sen et al., Human Skin Wounds: A Major and Snowballing Threat to Public Health and the Economy, Wound Repair Regen. 17(6): 763-771 (2009). The prevalence of pressure ulcers within inpatient settings has been reported to be 22%, with as many as 50-80% acquired within the hospital. Shahin et al., Pressure ulcer prevalence and incidence in intensive care patients: a literature review, Nurs Crit Care. 13(2):71-9 (2008). In addition, it is estimated that treated surgical wounds healing by secondary intention have a point prevalence of 4.1 per 10,000 population (95% confidence interval 3.5 to 4.7 per 10,000 population). Chetter et al., The epidemiology, management and impact of surgical wounds healing by secondary intention: a research programme including the SWHSI feasibility RCT, Southampton (UK): NIHR Journals Library; 2020 PMID: 32960518. Many of these wounds undergo treatments like negative pressure would therapy (with or without instillation). Lima et al., Negative pressure therapy for the treatment of complex wounds, Rev. Col. Bras. Cir. 44(1): 81-93 (2017). These wounds are in constant risk of infections. Clinically, wound assessment and diagnosis are based on laboratory testing, which is time consuming, labor intensive, costly, and does not consider the complex, changing wound environment. There is currently no simple way to quickly determine or monitor for a wound infection.

### SUMMARY

Accordingly, the present disclosure provides, in part, a device capable of contemporaneously detecting several pathogens in a matter of minutes. The device comprises electrochemical sensor array comprising a plurality of electrochemical sensors. Each electrochemical sensor is capable of detecting a different pathogen. The device may be fluidically connected to a biological fluid in an instrument such as a catheter bag, a urine collection bag, a colostomy bag, a wound dressing, or a wound exudate collection container to continuously monitor the presence, the absence of infection by one of several pathogens. Accordingly, the present disclosure provides a device, methods for detecting infection and methods of selecting appropriate therapy for the infection.

In one aspect, the current disclosure relates to a device for detecting an infection in a subject comprising an electrochemical sensor array, wherein electrochemical sensor array comprises a plurality of electrochemical sensors, wherein each electrochemical sensor comprises a working electrode, a reference electrode, and a counter electrode, wherein the electrochemical sensor array is fluidically connected to a wound exudate in a wound dressing or a wound exudate collection container, a wound exudate collection container of negative pressure wound therapy, a fluid collection container of negative pressure wound therapy with instillation, or urine in a catheter bag or a urine collection bag.

In embodiments, the electrochemical sensor array further comprises a sensor selected from a pH sensor and a temperature sensor. In embodiments, the electrochemical sensor array detects a change in pH, a change in temperature, an electrochemical reaction, binding to an aptamer, a change in color, or the combination of any two or more thereof. In embodiments, the device is capable of contemporaneously detecting at least two, or at least three, or at least 4 signals that are associated with individual pathogens. In embodiments, each electrochemical sensor is capable of independently performing an electrochemical measurement.

In embodiments, the device is capable of detecting an infection caused by a pathogen. In embodiments, the pathogen is selected a bacterium, a fungus and a parasite. In embodiments, the bacterium is selected from Pseudomonas aeruginosa, Proteus mirabilis, Escherichia coli, Klebsiella pneumoniae, other Klebsiella species, Staphylococcus aureus, Enterococcus faecalis, other Enterococcus species, Acinetobacter baumannii, Streptococcus group A species, Streptcoccus group B species, Staphylococcus epidermidis, Clostridium difficile, and Salmonella enterica. In embodiments, the fungus is Candida albicans. In embodiments, the parasite is Giardia, a fecal float worm, fecal roundworm and fecal flatworm.

In embodiments, the electrochemical sensor array is capable of contemporaneously detecting the presence or absence of at least two, or at least 3, or at least 4, or at least 5, or at least 6, or at least 7, or at least 8, or at least 9, or at least 10 pathogens. In embodiments, wherein the device detects a target molecule, and/or a metabolic activity of the pathogen. In embodiments, the target molecule is a quorum sensing molecule. In embodiments, the target molecule is a redox molecule. In embodiments, the target molecule is selected from quorum sensing molecules (without limitations, e.g., pyocyanin, E. coli autoinducer-2 (AI-2), N-Acyl Homoserine Lactones (AHL)), siderophores (without limitations, e.g. enterobactin, aerobactin, vibriobactin, salmochelin, pyoverdine, and pyochelin), cyclic signaling peptides (without limitations, e.g. Staphylococcus aureus autoinducing peptide (AIP), including AIP variants I to IV, and Enterococcus faecalis gelatinase biosynthesis activating pheromone (GBAP)), and autoinducers (without limitations, e.g. acylated homoserine lactones (AHLs), including N-(3-oxododecanoyl)-homoserine lactone and N-(butyryl)-homoserine lactone, 2-heptyl-3-hydroxy-4-quinolone (PQS), AIP variants I to IV). In embodiments, the target molecule is selected from 3O-C12-homoserine lactone (3OXO), putrescine, Shiga toxin, aerobactin, auto inducing peptide-1 (AIP-1), gelatinase biosynthesis activating peptide (GBAP), EppR (Protein), short hydrophobic peptide 3 SHP3 (also known as SHP1520), autoinducing peptide (AIP), autoinducing peptide 2 (AIP 2), pyocyanin, enterobactin, tyrosol and farnesol. In embodiments, the presence of the target molecule is indicative of a presence and/or an amount of and/or a number of viable cells of the pathogen.

In embodiments, the metabolic activity causes breakdown of a basic molecule. In embodiments, the metabolic activity is a urease activity. In embodiments, the metabolic activity changes pH of the urine or the wound exudate. In embodiments, the change in pH is an increase in pH.

In embodiments, the device is electrically connected or connectable to a reader. In embodiments, the reader provides an output of a presence and/or an amount of and/or a number of viable cells of a pathogen. In embodiments, the reader is capable of transmitting the specific signals to a display device. In embodiments, the signal is wirelessly transmitted.

In embodiments, the device detects the infection in less than one hour, less than 45 minutes, or less than 30 minutes, or less than 15 minutes, or less than 10 minutes, or less than 5 minutes, or less than 2 minutes, or less than 1 minute.

In one aspect, the current disclosure relates to a method of detecting an infection in urine of a subject, the method comprising (i) contacting urine from the subject with a device for detecting an infection, wherein the device comprises an electrochemical sensor array; and (ii) measuring a presence, absence or amount of one or more target molecule and/or a metabolic activity within the urine sample, wherein the target molecule and/or the metabolic activity is associated with the infection, wherein the electrochemical sensor array performs the measuring. In embodiments, the urine is collected in a catheter bag or a urine collection bag. In embodiments, the urinary tract infection is catheter associated urinary tract infections (CAUTI).

In one aspect, the current disclosure relates to a method of detecting a urinary tract infection in a subject, the method comprising (i) contacting urine from the subject with a device for detecting an infection, wherein the device comprises an electrochemical sensor array; and (ii) measuring a presence, absence or amount of one or more target molecule and/or a metabolic activity within the urine sample, wherein the target molecule and/or the metabolic activity is associated with the infection, wherein the electrochemical sensor array performs the measuring. In embodiments, the urine is collected in a catheter bag or a urine collection bag. In embodiments, the urinary tract infection is catheter associated urinary tract infections (CAUTI).

In one aspect, the current disclosure relates to a method of detecting a wound infection in a subject, the method comprising (i) contacting wound exudate from the subject with a device for detecting an infection, wherein the device comprises an electrochemical sensor array; and (ii) measuring a presence, absence or amount of one or more target molecule and/or a metabolic activity within the wound exudate, wherein the target molecule and/or the metabolic activity is associated with the infection, wherein the electrochemical sensor array performs the measuring. In embodiments, the wound exudate is collected in a wound dressing or a wound exudate collection container.

In one aspect, the current disclosure relates to a method of detecting a wound infection in a subject, the method comprising: (i) administering a dressing to a wound, optionally wherein the dressing comprises oxidized regenerated cellulose (ORC) and/or collagen, (ii) applying a negative pressure to the wound, (iii) collecting wound exudate in a wound exudate collection container, (iv) contacting wound exudate from a wound dressing or a wound exudate collection container with a device for detecting an infection, wherein the device comprises an electrochemical sensor array; and (v) measuring a presence, absence or amount of one or more target molecule and/or a metabolic activity within the wound exudate, wherein the target molecule and/or the metabolic activity is associated with the infection, wherein the electrochemical sensor array performs the measuring.

In one aspect, the current disclosure relates to a dressing comprising the device of any one of the embodiments disclosed herein, optionally wherein the dressing further comprises oxidized regenerated cellulose (ORC) and/or collagen.

In one aspect, the current disclosure relates to a urine collection bag comprising the device of any one of the embodiments disclosed herein.

In one aspect, the current disclosure relates to a catheter bag comprising the device of any one of the embodiments disclosed herein.

In one aspect, the current disclosure relates to a negative pressure wound therapy system comprising a wound dressing, and a negative pressure source and a wound exudate collection container, wherein wound exudate collection container comprises the device of any one of the embodiments disclosed herein, optionally wherein the dressing comprises oxidized regenerated cellulose (ORC) and/or collagen.

In one aspect, the current disclosure relates to a negative pressure wound therapy with installation system comprising a wound dressing, an instillation fluid, an instillation pump, and a negative pressure source and a wound exudate collection container, wherein wound exudate collection container comprises the device of any one of the embodiments disclosed herein, optionally wherein the dressing comprises oxidized regenerated cellulose (ORC) and/or collagen.

In one aspect, the current disclosure relates to a method of selecting a catheterized patient having or suspected as having a urinary tract infection for therapy, the method comprising: (i) contacting urine from a catheter bag or urine collection bag collected from a subject with a device of any one of the embodiments disclosed herein for detecting an infection, wherein the device comprises an electrochemical sensor array; (ii) measuring a presence, absence or amount of one or more target molecule and/or a metabolic activity within the urine sample, wherein the target molecule and/or the metabolic activity is associated with the infection; and (iii) selecting the patient for therapy with an appropriate antibiotic for the infection upon a positive test for infection.

In one aspect, the current disclosure relates to a method of selecting a patient having or suspected as having a wound infection for therapy, the method comprising: (i) contacting wound exudate from a wound dressing or a wound exudate collection container with a device of any one of the embodiments disclosed herein for detecting an infection, wherein the device comprises an electrochemical sensor array; (ii) measuring a presence, absence or amount of one or more target molecule and/or a metabolic activity within the wound exudate, wherein the target molecule and/or the metabolic activity is associated with the infection; and (iii) selecting the patient for therapy with an appropriate antibiotic for the infection upon a positive test for infection.

In one aspect, the current disclosure relates to a method of selecting a patient having or suspected as having a wound infection for therapy, the method comprising: (i) administering a dressing to a wound, optionally wherein the dressing comprises oxidized regenerated cellulose (ORC) and/or collagen, (ii) applying a negative pressure to the wound, (iii) collecting wound exudate in a wound exudate collection container, (iv) contacting wound exudate from a wound dressing or a wound exudate collection container with a device of any one of the embodiments disclosed herein for detecting an infection, wherein the device comprises an electrochemical sensor array; (v) measuring a presence, absence or amount of one or more target molecule and/or a metabolic activity within the wound exudate, wherein the target molecule and/or the metabolic activity is associated with the infection; and (vi) selecting the patient for therapy with an appropriate antibiotic for the infection upon a positive test for infection.

In one aspect, the current disclosure relates to a method for determining efficacy of a therapy in a catheterized patient receiving the therapy for a urinary tract infection, the method comprising (i) contacting urine from the subject receiving the therapy with a device of any one of the embodiments disclosed herein for detecting an infection, wherein the device comprises an electrochemical sensor array; (ii) measuring an amount of one or more target molecule and/or a metabolic activity within the urine sample, wherein the target molecule and/or the metabolic activity is associated with the infection; and (iii) comparing the amount of the target molecule and/or the metabolic activity with the amount of the target molecule and/or the metabolic activity prior to therapy, from another healthy subject, or a standard.

In one aspect, the current disclosure relates to a method for preventing a catheter-associated bacteremia or sepsis in a catheterized patient, the method comprising (i) contacting urine from the subject with a device of any one of the embodiments disclosed herein for detecting an infection, wherein the device comprises an electrochemical sensor array; (ii) measuring an amount of one or more target molecule and/or a metabolic activity within the urine sample, wherein the target molecule and/or the metabolic activity is associated with the infection; (iii) comparing the amount of the target molecule and/or the metabolic activity with the amount of the target molecule and/or the metabolic activity prior to therapy, from another healthy subject, or a standard and thereby detecting the presence of an infection; and (iv) administering therapy of an appropriate antibiotic for the infection.

In one aspect, the current disclosure relates to a method for determining efficacy of a therapy in a patient receiving the therapy for a wound infection, the method comprising (i) contacting wound exudate from the subject receiving the therapy with a device of any one of the embodiments disclosed herein for detecting an infection, wherein the device comprises an electrochemical sensor array; (ii) measuring a presence, absence or amount of one or more target molecule and/or a metabolic activity within the wound exudate, wherein the target molecule and/or the metabolic activity is associated with the infection; and (iii) comparing the amount of the target molecule and/or the metabolic activity with the amount of the target molecule and/or the metabolic activity prior to therapy, from another healthy subject, or a standard.

### BRIEF DESCRIPTION OF THE DRAWINGS

FIG. 1 shows a schematic representation of an electrochemical sensor array capable of contemporaneously detecting a plurality of pathogens. The listed pathogens are non-limiting illustrations.

FIG. 2A to FIG. 2C show graphical and schematic representations of illustrative non-limiting electrochemical sensors and their functioning. FIG. 2A shows an illustrative aptamer-based sensor, which works by measuring a peak current decrease upon enterobactin or Shiga toxin binding to the aptamer, due to the more constricted mobility of the aptamer and redox probe. FIG. 2B shows an illustrative pH sensor, which works by measuring the voltage at which a peak current occurs for a pH-sensitive redox molecule. An exemplary pH-sensitive redox molecule is methylene blue. The peak current voltage is observed at more negative values with an increase in pH. FIG. 2C shows an illustrative redox-active metabolite sensor, which works by the emergence of a peak current when a redox-active metabolite is present in solution. An illustrative redox-active metabolite is pyocyanin.

FIG. 3A shows a sensor array that was constructed with 4 electrodes, each electrode being specific for an individual pathogen's electrochemical test. FIG. 3B shows a schematic with a testing apparatus wired into a 50 mL Falcon tube.

FIG. 4A shows an embodiment of the current technology featuring a sensor array mounted inside a urine collection bag. The wires may be connected to a reader, which wirelessly transmits signal to an external device, such as an app on a wireless device such as a cellphone. The listed pathogens are non-limiting illustrations. FIG. 4B shows an embodiment of the current technology featuring a sensor array mounted on a wound dressing. FIG. 4C shows an embodiment of the current technology featuring a sensor array mounted inside a wound exudate collection container. FIG. 4D shows an embodiment of the current technology where wires routes out of a urine collection bag, a bandage or a wound exudate collection container. The wires may be connected to a reader. FIG. 4E shows an illustrative electrochemical sensor array capable of detecting the redox molecules or quorum sensing molecules (QSM), which are indicated by squares, diamonds, X and triangles, produced by Staphylococcus, E. coli, Pseudomonas and Klebsiella, which are diagrammatically shown in the bubble, representing a biological fluid in contact with the electrochemical sensor array. FIG. 4F shows the lack of signal when no redox label target is present, and a signal produced by a QSM target. FIG. 4G shows an exemplary readout produced by the electrochemical sensor array.

FIG. 5 shows the peak current in a Shiga toxin-specific aptamer-based sensor in the presence of increasing concentrations of Shiga toxin. As shown, Shiga toxin aptamer-based electrochemical sensor can detect 1 μM levels of Shiga toxin in urine samples.

FIG. 6A shows the evidence of successful inkjet printing of DNA on disposable printed circuit boards. DNA was printed on the disposable printed circuit boards was detected using UV light. FIG. 6B shows printing of kanamycin aptamer onto gold electrodes. FIG. 6C shows the raw data from measurement of current in the kanamycin aptamer-printed electrochemical sensors in the absence or presence of kanamycin. Scans in a blank solution were performed to gain a baseline measurement prior to introducing a target containing solution to gain the testing measurement. A comparison of measurements on each individual electrode are shown. FIG. 6D shows the normalized data from the measurements shown in FIG. 6C.

FIG. 7A to FIG. 7C show the detection of pyocyanin, a biomarker for Pseudomonas aeruginosa infection, in urine samples spiked with pyocyanin. FIG. 7A shows a photographs of the complete system of an osmotic system that was constructed for electrochemical measurements of pyocyanin. FIG. 7B shows a square-wave voltammetry (SWV) scan before and after 40 min of osmotic concentration for urine spiked with 1 μM pyocyanin. FIG. 7C shows that PBS, saliva (SAL), and urine (URI) spiked with 1 μM pyocyanin showed the pyocyanin peak current increased by 350-400% after 40 min of forward osmosis.

FIG. 8 shows the detection of Pseudomonas aeruginosa urine samples using the electrochemical sensors disclosed herein.

### DETAILED DESCRIPTION

The present disclosure provides, in part, a device capable of contemporaneously detecting several pathogens in a matter of minutes. The device comprises electrochemical sensor array comprising a plurality of electrochemical sensors. Each electrochemical sensor is capable of detecting a different pathogen. This device is potentially very useful for identifying urinary tract infection in catheterized individuals, or to detect a wound infection.

Urinary tract infections (UTIs) are one of the most common health care acquired infections in the US. Up to 70-80% of these infections can be attributed to the use of an indwelling urinary catheter. For patients in long term care (LTC) facilities with indwelling urinary catheters (IUC), the risk of developing CAUTI is time-dependent, increasing at a rate of 3% to 8% each day of IUC use, reaching 100% prevalence at 30 days. Complications from untreated catheter associated urinary tract infections (CAUTI) include more serious bladder and kidney infections, which can lead to bacteremia and sepsis.

There is currently no simple way to quickly determine or monitor for a catheter associated urinary tract infection or impending catheter blockage. A urinalysis can be performed by a trained technician if the urine appears cloudy, yet this type of test only indicates the presence of blood cells, which may be present due to other issues not relevant to a urinary tract infection. Culturing the urine to determine a bacterial infection takes multiple days, by which point the patient is likely suffering from discomfort and potentially further complications. In many cases, an antimicrobial will be prescribed before positive diagnosis of a UTI, even though a UTI may not exist, and is especially prevalent in nursing home residents with advanced dementia that ultimately do not meet minimum criteria for antimicrobial initiation. The over-prescription and misuse of antibiotics is the leading cause of antimicrobial resistance according to the World Health Organization (Antibiotic Resistance, 2018). A simple, cheap, and effective way to continuously monitor for bacterial infections in catheterized patients would help prevent these issues and result in better patient outcomes

The device disclosed herein is useful for preventing bacteremia and sepsis based on quick identification of catheter associated urinary tract infections (CAUTI).

The present disclosure is based, in part, on the discovery that multiple electrochemical sensor arrays may be combined in a device to contemporaneously, and quickly determine presence of multiple pathogens in a biological sample. Provided herein is a device capable of contemporaneously detecting several pathogens in a matter of minutes. The device comprises electrochemical sensor array comprising a plurality of electrochemical sensors. Each electrochemical sensor is capable of detecting a different pathogen.

Provided herein is a device capable of contemporaneously detecting several pathogens via an electrochemical sensor array. In embodiments, the device is capable of analyzing urine in a collection bag or a catheter bag during use, or analyzing wound exudate in a dressing or wound exudate collection container without the need for sample collection or laboratory tests. In embodiments, the device is capable of contemporaneously detecting the presence and/or the amount of and/or the number of viable cells of a plurality of pathogens. In embodiments, the ability to detect and quantify the level of bacterial infections provides a guidance for specific treatment, fewer complications, and limit the spread of bacterial infections to the bladder and kidneys that can lead to more extreme complications like sepsis.

In one aspect, the current disclosure relates to a device for detecting an infection in a subject comprising an electrochemical sensor array, wherein electrochemical sensor array comprises a plurality of electrochemical sensors, wherein each electrochemical sensor comprises a working electrode, a reference electrode, and a counter electrode, wherein the electrochemical sensor array is fluidically connected to a wound exudate in a wound dressing or a wound exudate collection container, a wound exudate collection container of negative pressure wound therapy, a fluid collection container of negative pressure wound therapy with instillation, or urine in a catheter bag or a urine collection bag.

### Electrochemical Sensor Array

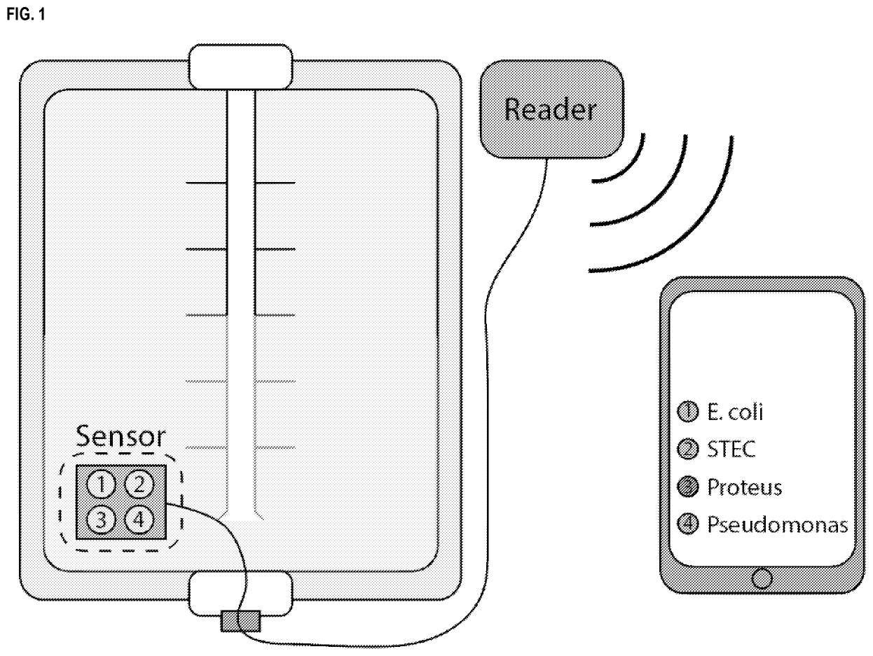

In embodiments, the device for detecting an infection in a subject comprising an electrochemical sensor array, wherein electrochemical sensor array comprises a plurality of electrochemical sensors. A schematic representation of a device of present disclosure is shown in FIG. 1. In the non-limiting embodiment of FIG. 1, the device is connected in-line with a urine collection bag. In this non-limiting example, the sensor reader is designed to wirelessly transmit the results to a smart phone app, which depicts which, if any, of the pathogens are present, along with their infection levels. In embodiments, the device comprising an electrochemical sensor array is embedded inside a catheter collection bag. In embodiments, the device is capable of contemporaneously detecting the presence and/or the amount of and/or the number of viable cells of uropathogenic bacteria in human urine samples. In embodiments, the device comprising an electrochemical sensor array is embedded inside other instruments such as a urine collection bag, a catheter bag, a wound dressing, and a wound exudate collection bag.

In embodiments, the device is capable of continuous and contemporaneous monitoring of the presence and/or the amount of and/or the number of viable cells of multiple pathogens. In embodiments, the device is capable of providing rapid, sensitive, simple to use, and inexpensive diagnosis of urinary tract infection, wound infection, etc. for example, in embodiments, the device is capable of detecting some of the most common bacteria found in CAUTI, such as Escherichia coli, Proteus mirabilis, and Pseudomonas aeruginosa. In embodiments, these bacterial species may be rapidly identified by detecting and quantifying specific metabolites that the bacteria produce using electrochemical voltammetry methods. In embodiments, the metabolites are secreted. In embodiments, the detection by the device disclosed herein is faster and easier than culturing or PCR-based techniques. In embodiments, the instrumentation for running the electrochemical tests along with the sensors consists of very simple and low-cost electronics and materials. In embodiments, In embodiments, the detection by the device disclosed herein has less false-negative rates compared to other rapid UTI detection methods such as dip-sticks.

The secreted metabolites are present in much larger quantities in the urine compared to the number of actual bacterial cells.

In embodiments, the device disclosed herein combines several specific and individual tests into one simple multiplexed disposable sensor that is capable of specifically detect four different pathogens simultaneously. In embodiments, the device disclosed herein is a miniature product. In embodiments, the device disclosed herein is a battery-powered sensor array reader with WI-FI capabilities that powers the sensors in the urine collection bag of bedridden patients and transmits warning signals to caregivers. In embodiments, the device disclosed herein comprises an USB tethered reader that is capable of transmitting test results. In embodiments, the device disclosed herein is capable of wirelessly transmitting test results a cellular phone.

In embodiments, the devise will further comprise a reader. In embodiments, the devise is calibrated with spiked samples. In embodiments, the calibration comprises bacterial counts to signal response. In embodiments, a calibrated device is capable of quantification the level of infection. In embodiments, a calibrated device is capable of determining true infections versus non-threatening asymptomatic or background bacterial presence.

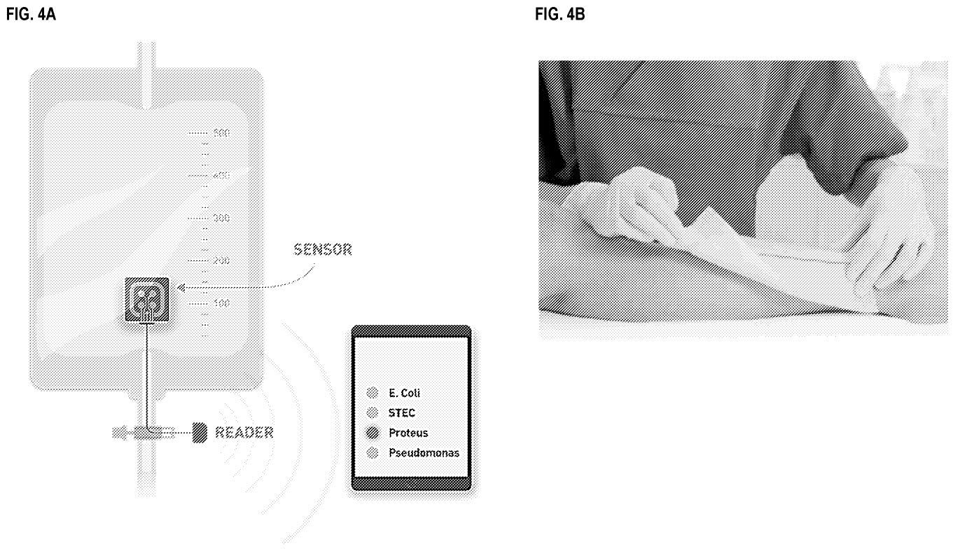

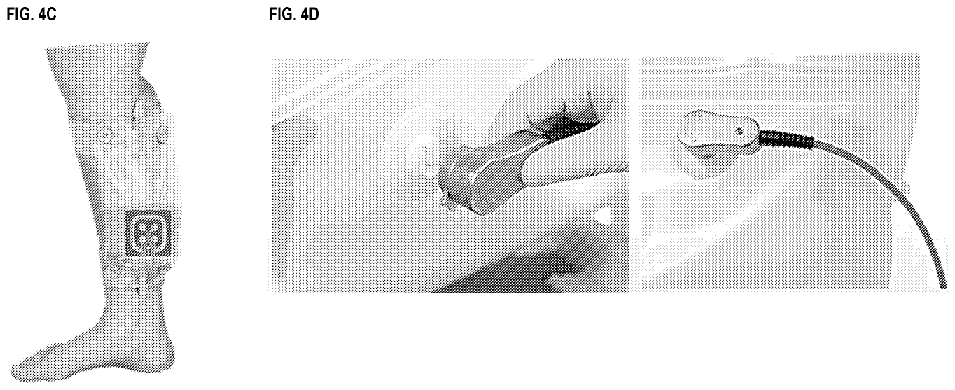

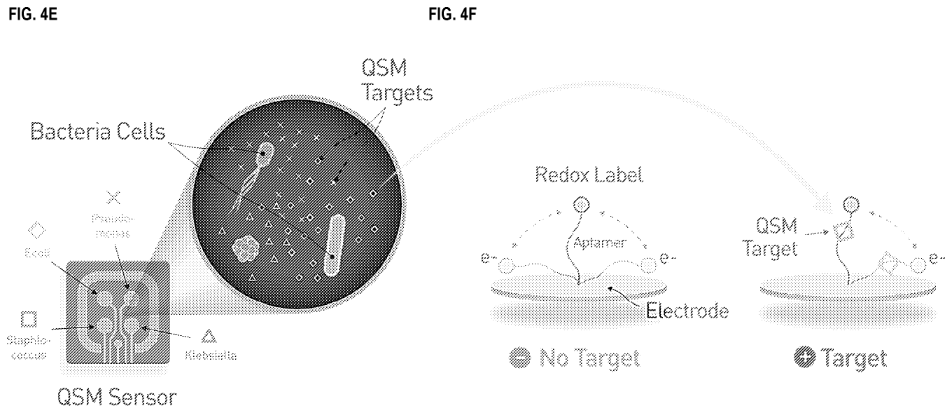

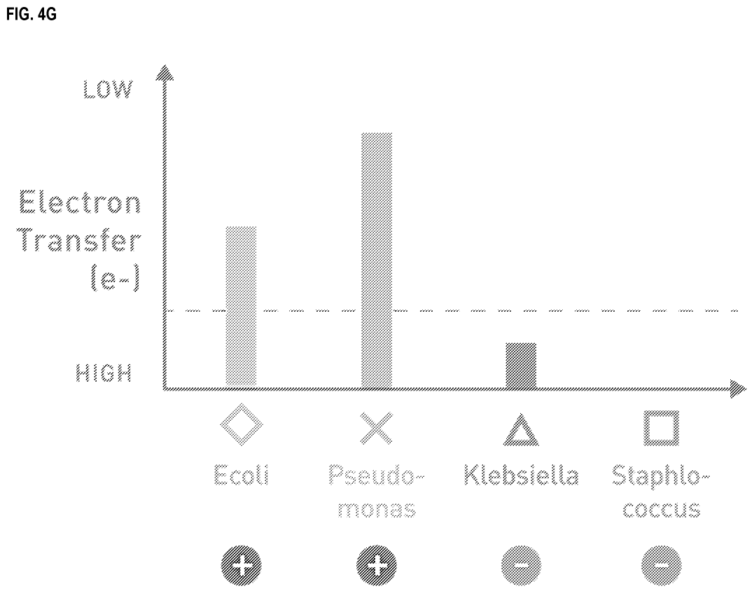

An illustrative embodiments of the electrochemical sensor array is shown in FIG. 4A to FIG. 4C. As shown in FIG. 4A, the device disclosed herein may be mounted inside a urine collection bag. In embodiments, the wires may be connected to a reader, which wirelessly transmits signal to an external device, such as an app on a wireless device such as a cellphone. FIG. 4B shows an illustrative embodiment of the current technology featuring a sensor array mounted on a wound dressing. FIG. 4C shows an illustrative embodiment of the current technology featuring a sensor array mounted inside a wound exudate collection container. In any of the embodiments disclosed here, wires may route out of the urine collection bag, the bandage or the wound exudate collection container with a device mounted in it, as shown in FIG. 4D. in embodiments, the wires may be connected to a reader.

FIG. 4D to FIG. 4G illustrative function of the electrochemical sensor array. FIG. 4E shows an illustrative electrochemical sensor array capable of detecting the redox molecules or quorum sensing molecules (QSM), which are indicated by squares, diamonds, x and triangles, produced by Staphylococcus, E. coli, Pseudomonas and Klebsiella, which are diagrammatically shown in the bubble, representing a biological fluid in contact with the electrochemical sensor array. QSMs are unique to each bacteria. In embodiments, the electrochemical sensor-based test for a given QSM is specific for the bacterium that produces it. QSMs are produced in huge quantity, and in quantities proportionate to the cell number of the bacterium producing it. In embodiments, the electrochemical sensor-based test for a given QSM detects the presence and/or the amount of and/or the number of viable cells of the bacterium producing the QSM. QSMs are only present during infections. In embodiments, the electrochemical sensor-based test for a given QSM produces a signal only when infection is present. Thus, in embodiments, the electrochemical sensor-based test for a given QSM may be used to determine administration of a treatment, continuation of the treatment and/or efficacy of the treatment. In embodiments, QSMs are detected based on the electrochemical signature of the QSM. In embodiments, a QSM is detected based on binding of the QSM to an aptamer. Aptamers are specific to each QSM. FIG. 4F shows the lack of signal when no redox label target is present, and a signal produced by a QSM target. FIG. 4G shows an exemplary readout produced by the electrochemical sensor array.

The Table below shows exemplary non-limiting target molecules and pathogens that may be detected using electrochemical sensor arrays in a device disclosed here:

| Bacteria | Target Molecule | Chemical Structure |
|---|---|---|
| Pseudomonas aeruginosa | 3O-C12-Homoserine Lactone (3OXO) | 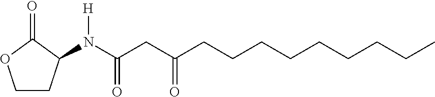 [SMILES: CCCCCCCCCC(=O)CC(=O)N[C@H]1CCOC1=O] |
| Proteus mirabilis | putrescine | 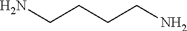 |
| Escherichia coli | Shiga toxin | 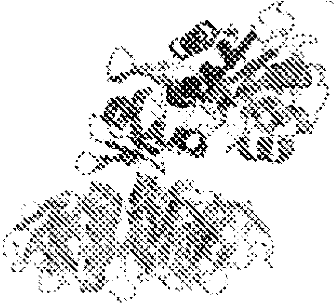 |
| Klebsiella pneumonia | aerobactin | 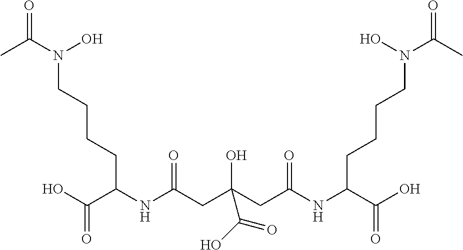 [SMILES: CC(=O)N(O)CCCCC(NC(=O)CC(O)(CC(=O)NC(CCCCN(O)C(C)=O)C(=O)O)C(=O)O)C(=O)O] |
| Staphylococcus aureus | Auto Inducing Peptide-1 (AIP-1) | 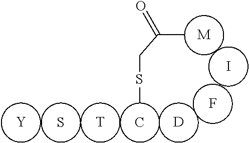 |
| Enterococcus faecalis | Gelatinase Biosynthesis Activating Peptide (GBAP) | 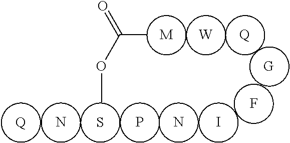 |
| Acinetobacter | EppR (Protein) | |
| baumannii | | |
| Streptococcus | Short Hydrophobic | DILIIVGG |
| agalactiae | Peptide 3 SHP3 (also | |
| | known as SHP1520) | |
| Staphylococcus | AIP | RIPTSTGFF |
| pseudintermedius | | |
| Staphylococcus | AIP 2 | DSVCASYF |
| epidermidis | | |
| Uropathogenic Escherichia coli | Enterobactin | 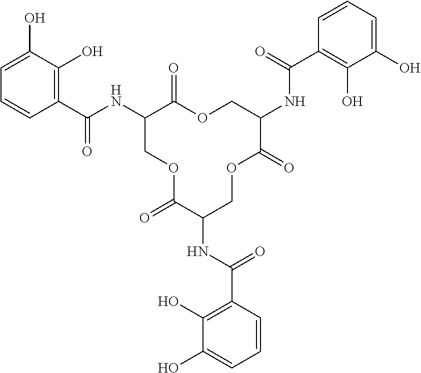 [SMILES: O=C(NC1COC(=O)C(NC(=O)c2cccc(O)c2O)COC(=O)C(NC(=O)c2cccc(O)c2O)COC1=O)c1cccc(O)c1O] |

The Table below shows additional exemplary non-limiting target molecules and as applicable, pathogens being detected, that may be detected using electrochemical sensor arrays in a device disclosed here:

| Target Molecule | Notes |
|---|---|
| calprotectin | fecal samples |
| farnesol | Candida albicans |
| tyrosol | Candida albicans |
| toxin B | C. diff issue |
| tnf-alpha | sub picomolar detection limit needed |
| auto-inducer 2 | general bacterial marker |
| cholerae autoinducer 1 | Vibrio cholerae |
| 4,5-dihydroxy-2,3-pentanedione | Salmonella enterica |
| (DPD) | |

In embodiments, the electrochemical sensor comprises a working electrode, a reference electrode, and a counter electrode.

In embodiments, the working electrode material of one or more sensors is selected from gold (Au), silver (Ag), platinum (Pt), indium tin oxide (ITO), carbon, multi-walled carbon nanotubes, single-walled carbon nanotubes, carbon nanofibers, graphene, carbon-platinum composites, multi-walled carbon nanotubes with gold nanoparticles, and any combination thereof. In embodiments, the working electrode has a diameter between about 0.1 mm and about 10 mm, optionally between about 1 mm and about 5 mm. In embodiments, the working electrode has a diameter between about 1.5 mm and about 4 mm.

In an illustrative embodiment, the working electrode comprises about 1.5 mm gold screen-printed at elevated temperature. In another illustrative embodiment, the working electrode comprises about 1.5 mm platinum. In another illustrative embodiment, the working electrode has electrodeposition of gold to coat copper electrodes exposed on a printed circuit board. In another illustrative embodiment, the working electrode has screen-printed carbon paste to coat copper electrodes exposed on a printed circuit board. In another illustrative embodiment, the working electrode comprises about 4 mm gold. In yet another illustrative embodiment, the working electrode comprises about 1.5 mm Au screen-printed at low temperature. In yet another illustrative embodiment, the device may comprise an oxidizing and a reducing working electrode, for amplifying the signal, and the two working electrodes consist of gold and or platinum.

In embodiments, the working electrodes may make up a wall or part of a wall of a channel, such as a microfluidic channel or a nanofluidic channel, into which the fluid sample is introduced and within which the redox reaction takes place. In embodiments, the oxidizing electrode and the reducing electrode are separated by a distance of about 20 nm to 1 mm or greater. In embodiments, the distance between the oxidizing electrode and the reducing electrode is from 20 nm to about 100 nm, or from about 20 nm to about 40 nm, or from about 40 nm to about 60 nm, or from about 60 nm to about 80 nm, or from about 80 nm to about 100 nm, or from about 100 nm to about 150 nm, or from or from about 50 nm to about 500 nm, or from about 100 nm to about 1 μm, or from about 500 nm to about 5 μm, or from about 1 μm to about 10 μm, or from about 5 μm to about 50 μm, or from about 10 μm to about 100 μm, or from about 50 μm to about 500 μm, or from about 100 μm to about 1 mm, or greater. In embodiments, the distance between the oxidizing electrode and the reducing electrode is from 20 nm to about 100 nm, or from about 20 nm to about 40 nm, or from about 40 nm to about 60 nm, or from about 60 nm to about 80 nm, or from about 80 nm to about 100 nm, or from about 100 nm to about 150 nm.

The surface area of the working electrodes can be selected to accommodate a desired size of the device. Without being bound by theory, larger surface area generally improves the signal and sensitivity of the device. For example, in different embodiments, the surface area of each working electrode can be about 100, about 200, about 300, about 400, about 500, about 800, about 1000, about 2000, about 3000, about 5000, about 10000, about 50000, about 100000, about 200000, or about 500000 nm², or about 1, about 2, about 5, about 10, about 50, about 100, about 200, about 300, about 400, about 500, about 800, about 1000, about 2000, about 3000, about 5000, about 10000, about 50000, about 100000, about 200000, or about 500000 μm², or about 1, about 2, about 4, about 7 mm² or greater. In different embodiments, the surface area of each working electrode can be about 100, about 200, about 300, about 400, about 500, about 800, about 1000, about 2000, about 3000, about 5000, about 10000, about 50000, about 100000, about 200000, or about 500000 nm², or about 1, about 2, about 5, about 10 μm², or greater.

Any reference electrode that is compatible with the chosen working electrode may be used. In embodiments, the reference electrode material of one or more sensors is selected from silver (Ag), silver chloride (AgCl), and platinum (Pt). In embodiments, the reference electrode comprises silver (Ag). In embodiments, the reference electrode comprises Ag/AgCl.

In embodiments, the electrochemical sensor further comprises a counter electrode. In embodiments, the counter electrode of each sensor is identical to the working electrode.

In some embodiments, the electrochemical sensor is a microfluidic sensor comprises a working electrode, a counter electrode and a reference electrode. In these embodiments, the current flows the current flows through the working electrode and the counter electrode. In some embodiments, the counter electrode functions as a cathode and the working electrode is operating as an anode. In alternative embodiments, the counter electrode functions as an anode and the working electrode is operating as a cathode. In some embodiments, the counter electrode has a surface area much larger than that of the working electrode.

In embodiments, the electrochemical sensor may be used for measuring an electrochemical reaction taking place at the working electrode at a well-defined potential. In embodiments, the electrochemical reaction taking place at the working electrode is measured in comparison to the electrochemical reaction taking place at the reference electrode. In embodiments, the electrochemical sensor may be used for measuring the redox peak present at a given potential, which is sensitive to pH of the solution. In embodiments, the redox peak present at a given potential at the working electrode is measured in comparison to the redox peak present at the reference electrode.

In embodiments, the electrochemical sensor may be used for measuring the binding of a molecule to an aptamer, causing a change in electrical current passing through the electrode. In embodiments, the change in electrical current passing through the electrode working electrode is measured in comparison to the electric current at the reference electrode.

The Table below shows exemplary non-limiting aptamer sequences that may be used for detecting the presence, absence or amount of Shiga in the electrode electrochemical sensor arrays in a device disclosed here. The sequences of the aptamers are indicated in boldface-underlined font.

| Target | |
|---|---|
| Molecule | Aptamer sequence |
| Shiga | (+)ACCCCTGCATCCTTTGCTGGGGTAACTAGCATTCATTTCCCACACCCGTCCCGTCCATATAGT |
| | CTAGAGGGCCCCAGAAT (SEQ ID NO: 1) |
| Shiga | (−)ATTCTGGGGCCCTCTAGACTATATGGACGGGACGGGTGTGGGAAATGAATGCTAGTTACC |
| | CCAGCAAAGGATGCAGGGGT (SEQ ID NO: 2) |

In embodiments, the aptamers are generated using a DNA capture element sensing system. In embodiments, the aptamers are selected by conventional SELEX. See e.g. Fan et al., Aptamer Selection Express: A Novel Method for Rapid Single-Step Selection and Sensing of Aptamers, Journal of Biomolecular Techniques 19:311-321 (2018).

In embodiments, highly enriched unmodified RNA aptamer pools may be cloned, and ˜100 clones from each pool may be sequenced. In embodiments, the individual clones may be classified into groups from m-SELEX and groups from p-SELEX based on the alignments of individual aptamer sequences. For example, in embodiments, m-SELEX sequences may be grouped into groups mA, mB, mC, mD, etc., based on sequence alignment. Similarly, in embodiments, p-SELEX sequences may be grouped into groups pA, pB, pC, pD, etc., based on sequence alignment. The Table below shows exemplary non-limiting m-SELEX sequences and p-SELEX sequences of RNA aptamers that are classified as above. See e.g., Challa et al., Selective Evolution of Ligands by Exponential Enrichment to Identify RNA Aptamers against Shiga Toxins, Journal of Nucleic Acids, 2014: 214929 (2014). The sequence of 19 bases is identical among groups mA, mB, pA and pC are indicated by underline. In embodiments, these sequences may be used for detecting the presence, absence or amount of Shiga in the electrode electrochemical sensor arrays in a device disclosed here.

| Group | Sequence |
|---|---|
| mA | ATTAGCTATCTTCCACGATTCGATCAGGCAGTACGTCGT (SEQ ID NO: 3) |
| mB | ACAGTTATCCGACTGCTATTCGATCAGGCAGTACGTAGC (SEQ ID NO: 4) |
| mC | CAGGCTGTTCTGACGCATAAGGAATGCGCTGTTGCAGAG (SEQ ID NO: 5) |
| mD | TTGGTCCTGCTTTGGATAGTCGCGAAAGGGGTGCCACTG (SEQ ID NO: 6) |
| m-Singles | Orphan sequences |
| pA | ACAGTTATCCGACTGCTATTCGATCAGGCAGTACGTAGC (SEQ ID NO: 7) |
| pB | ACCGAGCGGTTTTACGTCTCAAGTAGTATCCCGTTTTGC (SEQ ID NO: 8) |
| pC | ATTAGCTATCTTCCACGATTCGATCAGGCAGTACGTCGT (SEQ ID NO: 9) |
| pD | TTGCCATCCTGTACTATGCTCTATCGGGGGGTTTAGTGATCCTTCGTCCAACTATC |
| | (SEQ ID NO: 10) |
| p-Singles | Orphan sequences |

Orphan sequences are the sequences that are seen only in one isolate.

In embodiments, the electrochemical sensor thereby facilitates an electrochemical detection of a predetermined redox-active compound associated with the infection. In embodiments, the electrochemical detection of the predetermined redox-active compound associated with the infection (without limitation, e.g., pyocyanin) and thereby detect the presence of a specific pathogen (without limitation, e.g., Pseudomonas aeruginosa). In embodiments, the defined potential of the working electrode may be varied, and the response from the electrochemical reaction is seen from the current of the working electrode. In embodiments, the electrochemical sensor comprises a second working electrode. In embodiments, the second working electrodes with respect to one or more of surface area, size, material, and coating. In embodiments, the electrochemical sensor may include an oxidizing working electrode and a reducing working electrode. In embodiments, the concentration of a target molecule and/or a metabolic activity associated with the infection is measured as current flow through the oxidizing electrode and the reducing electrode. In embodiments, the working electrode is one of an oxidizing electrode and a reducing electrode, and the second working electrode is the other of the oxidizing electrode and the reducing electrode. A potential suitable for oxidizing the target molecule and/or the metabolic activity associated with the infection is applied at the oxidizing electrode and a potential suitable for reducing the target molecule and/or the metabolic activity associated with the infection is applied at the reducing electrode.

In embodiments, a given target molecule and/or a metabolic activity associated with the infection electrochemically reacts differently on different electrode surfaces. Thus, different electrode materials and geometries used for chemical detection will give different results. Accordingly, in embodiments, the sensor array increases the sensitivity and specificity of the measurement and reduces the noise from other substances present in a biological sample. In embodiments, the sensor array may comprise two or more sensors, wherein each sensor comprises a working electrode that differs from the other working electrodes with respect to at least one of the following characteristics: surface area, size, material, and coating.

The electrochemical measurement can be made in any suitable manner. In embodiments, the electrochemical measurement may made by squarewave voltammetry, linear sweep voltammetry, staircase voltammetry, cyclic voltammetry, normal pulse voltammetry, differential pulse voltammetry, and chronoamperometry. In embodiments, the electrochemical measurement is square wave voltammetry and the current flow is measured in response to one or more square wave potentials. In embodiments, optionally cyclic voltammetry is used and the working electrode potential is ramped linearly versus time. In embodiments, the potential is ramped linearly up, and when a set potential is reached, the potential is ramped in the opposite direction to the initial potential, and the cycle is repeated. In embodiments, the working electrode potential include linear sweep voltammetry, staircase voltammetry, square-wave voltammetry, and differential pulse voltammetry.

In embodiments, the presence, absence or amount of the compound is measured as current flow through the working electrode. In embodiments, the presence, absence or amount of compound is measured as current flow through the oxidizing electrode and the reducing electrode.

In embodiments, the electrochemical sensor array disposable. In embodiments, the sensor array is integrated inside of the sterile bag. In embodiments, a single wire exits through the drainage cap to connect to a reader, optionally, the reader is battery powered. In embodiments, similarly to currently sensor array comprises integrated temperature and conductance sensors.

In one aspect, the current disclosure relates to a device for detecting an infection in a subject comprising an electrochemical sensor array, wherein electrochemical sensor array comprises a plurality of electrochemical sensors, wherein each electrochemical sensor comprises a working electrode, a reference electrode, and a counter electrode, wherein the electrochemical sensor array is fluidically connected to a wound exudate in a wound dressing or a wound exudate collection container, a wound exudate collection container of negative pressure wound therapy, a fluid collection container of negative pressure wound therapy with instillation, or urine in a catheter bag or a urine collection bag.

In embodiments, the electrochemical sensor array further comprises a sensor selected from a pH sensor and a temperature sensor. In embodiments, the electrochemical sensor array detects a change in pH, a change in temperature, an electrochemical reaction, binding to an aptamer, a change in color, or the combination of any two or more thereof. In embodiments, the device is capable of contemporaneously detecting at least two, or at least three, or at least 4, or at least 5, or at least 6, or at least 7, or at least 8, or at least 9, or at least 10, or at least 11, or at least 12, or at least 13, or at least 14, or at least 15, or at least 16 signals. In embodiments, the signal is associated with a pathogen. In embodiments, each electrochemical sensor is capable of independently performing an electrochemical measurement. In embodiments, the electrochemical measurement is selected from square wave voltammetry, linear sweep voltammetry, staircase voltammetry, cyclic voltammetry, normal pulse voltammetry, differential pulse voltammetry, and chronoamperometry. In embodiments, the electrochemical measurement is square wave voltammetry. In embodiments, the electrochemical measurement is measurement of a current flow. In embodiments, the current flow is measured in response to one or more square wave potentials.

In embodiments, the working electrode is comprised of gold (Au), silver (Ag), platinum (Pt), indium tin oxide (ITO), carbon, carbon nanotubes, carbon nanofibers, graphene, carbon-platinum composites, carbon nanotubes with gold nanoparticles, or any combination thereof. In embodiments, the electrochemical sensors further comprise a reference electrode, optionally wherein the reference electrode is comprised of silver (Ag), silver chloride (AgCl), and platinum (Pt), and any combination thereof. In embodiments, each electrochemical sensor comprises a second working electrode. In embodiments, the second working electrode is comprised of gold (Au), silver (Ag), platinum (Pt), indium tin oxide (ITO), carbon, carbon nanotubes, carbon nanofibers, graphene, carbon-platinum composites, carbon nanotubes with gold nanoparticles, or any combination thereof. In embodiments, the working electrode is one of an oxidizing electrode and a reducing electrode, and the second working electrode is the other of the oxidizing electrode and the reducing electrode.

In embodiments, the device is capable of detecting an infection caused by a pathogen. In embodiments, the pathogen is selected a bacterium, a fungus and a parasite. In embodiments, the bacterium is selected from Pseudomonas aeruginosa, Proteus mirabilis, Escherichia coli, Klebsiella pneumoniae, other Klebsiella species, Staphylococcus aureus, Enterococcus faecalis, other Enterococcus species, Acinetobacter baumannii, Streptococcus group A species, Streptococcus Group B species, Staphylococcus epidermidis, Pseudomonas aeruginosa, Clostridium difficile, and Salmonella enterica. In embodiments, the fungus is Candida albicans. In embodiments, the parasite is Giardia, a fecal float worm, fecal roundworm and fecal flatworm.

In embodiments, the electrochemical sensor array is capable of contemporaneously detecting the presence or absence of at least two, or at least 3, or at least 4, or at least 5, or at least 6, or at least 7, or at least 8, or at least 9, or at least 10 pathogens. In embodiments, wherein the device detects a target molecule, and/or a metabolic activity of the pathogen. In embodiments, the target molecule is a quorum sensing molecule. In embodiments, the target molecule is a redox molecule.

In embodiments, the metabolic activity causes breakdown of a basic molecule. In embodiments, the metabolic activity is a urease activity. In embodiments, the metabolic activity changes pH of the urine or the wound exudate. In embodiments, the change in pH is an increase in pH. In embodiments, the metabolic activity is a bacterial urease activity. In embodiments, the bacterial urease activity generates ammonia from the urea, and thereby increasing the pH. Therefore, in embodiments, the urease activity of a bacterium (without limitation, e.g. Proteus) makes the biological fluid (without limitation, e.g. urine) alkaline, thereby allowing the detection of the presence or absence of an infection by the bacterium. It is noted that the alkaline conditions stimulate the formation of crystals of calcium and magnesium phosphate and the development of a crystalline bio film, which eventually blocks the flow of urine from the bladder. In embodiments, the device detects the presence, absence or amount of the urease activity of the pathogen.

In embodiments, the device detects the presence, absence or amount of the target molecule and/or the metabolic activity and/or the metabolic activity of the pathogen. In embodiments, the presence, absence or amount of the target molecule and/or the metabolic activity is measured as current flow through the working electrode. In embodiments, the presence, absence or amount of the target molecule and/or the metabolic activity is measured as current flow through the oxidizing electrode and the reducing electrode.

In embodiments, the presence, absence or amount of the target molecule and/or the metabolic activity is measured as a change in pH. In embodiments, the target molecule is selected from quorum sensing molecules (without limitations, e.g., pyocyanin, E. coli autoinducer-2 (AI-2), N-Acyl Homoserine Lactones (AHL)), siderophores (without limitations, e.g. enterobactin, aerobactin, vibriobactin, salmochelin, pyoverdine, and pyochelin), cyclic signaling peptides (without limitations, e.g. Staphylococcus aureus autoinducing peptide (AIP), including AIP variants I to IV, and Enterococcus faecalis gelatinase biosynthesis activating pheromone (GBAP)), and autoinducers (without limitations, e.g. acylated homoserine lactones (AHLs), including N-(3-oxododecanoyl)-homoserine lactone and N-(butyryl)-homoserine lactone, 2-heptyl-3-hydroxy-4-quinolone (PQS), AIP variants Ito IV). In embodiments, the target molecule is selected from 3O-C12-homoserine lactone (3OXO), putrescine, Shiga toxin, aerobactin, auto inducing peptide-1 (AIP-1), gelatinase biosynthesis activating peptide (GBAP), EppR (Protein), short hydrophobic peptide 3 SHP3 (also known as SHP1520), autoinducing peptide (AIP), autoinducing peptide 2 (AIP 2), pyocyanin, enterobactin, tyrosol and farnesol. In embodiments, the presence of the target molecule is indicative of a presence and/or an amount of and/or a number of viable cells of the pathogen. In embodiments, the pathogen is selected from Pseudomonas aeruginosa, Proteus mirabilis, Escherichia coli, Klebsiella pneumoniae, other Klebsiella species, Staphylococcus aureus, Enterococcus faecalis, other Enterococcus species, Acinetobacter baumannii, Streptococcus group A species, Streptcoccus group B species, Staphylococcus epidermidis, Clostridium difficile, Salmonella enterica, Candida albicans, Cryptococcus neoformans, and Aspergillus species.

In embodiments, the presence, absence or amount of: 3O-C12-homoserine lactone (3OXO) is indicative of the presence and/or the amount of and/or the number of viable cells of Pseudomonas aeruginosa, putrescine is indicative of the presence and/or the amount of and/or the number of viable cells of Proteus mirabilis, Shiga toxin is indicative of the presence and/or the amount of and/or the number of viable cells of Escherichia coli, aerobactin is indicative of the presence and/or the amount of and/or the number of viable cells of Klebsiella and/or E. coli, auto inducing peptide-1 (AIP-1) is indicative of the presence and/or the amount of and/or the number of viable cells of Staphylococcus aureus, gelatinase biosynthesis activating peptide (GBAP) is indicative of the presence and/or the amount of and/or the number of viable cells of Enterococcus faecalis, EppR protein is indicative of the presence and/or the amount of and/or the number of viable cells of Acinetobacter baumannii, short hydrophobic peptide 3 SHP3 (SHP1520) is indicative of the presence and/or the amount of and/or the number of viable cells of Streptococcus agalactiae, autoinducing peptide (AIP) is indicative of the presence and/or the amount of and/or the number of viable cells of Staphylococcus pseudintermedius, autoinducing peptide 2 (AIP 2) is indicative of the presence and/or the amount of and/or the number of viable cells of Staphylococcus epidermidis, pyocyanin is indicative of the presence and/or the amount of and/or the number of viable cells of Pseudomonas aeruginosa, tyrosol is indicative of the presence and/or the amount of and/or the number of viable cells of Candida albicans, farnesol is indicative of the presence and/or the amount of and/or the number of viable cells of Candida albicans, and/or enterobactin is indicative of the presence and/or the amount of and/or the number of viable cells of uropathogenic Escherichia coli.

In embodiments, the device is electrically connected or connectable to a reader. In embodiments, the reader provides an output of a presence and/or an amount of and/or a number of viable cells of a pathogen. In embodiments, the reader is capable of transmitting the specific signals to a display device. In embodiments, the display device is a hand-held device. In embodiments, the display device is a portable device. In embodiments, the signal is wirelessly transmitted. In embodiments, the pathogen is selected from Pseudomonas aeruginosa, Proteus mirabilis, Escherichia coli, Klebsiella pneumoniae, other Klebsiella species, Staphylococcus aureus, Enterococcus faecalis, other Enterococcus species, Acinetobacter baumannii, Streptococcus group A species, Streptcoccus group B species, Staphylococcus epidermidis, Clostridium difficile, Salmonella enterica, Candida albicans, Cryptococcus neoformans, and Aspergillus species.

In embodiments, the device detects the infection in less than one hour, less than 45 minutes, or less than 30 minutes, or less than 15 minutes, or less than 10 minutes, or less than 5 minutes, or less than 2 minutes or less than 1 minute.

In one aspect, the current disclosure relates to a dressing comprising the device of any one of the embodiments disclosed herein, optionally wherein the dressing further comprises oxidized regenerated cellulose (ORC) and/or collagen.

Some current attempts to monitor urine collection bags include the measurement of pH change via a color change reaction (Milo 2016). However, color-based tests are limited in use due to the requirement of a visual inspection and interpretation of the color change as well as not providing any multiplexed detection. Other collection bag monitoring technologies only measure urine flow or urine levels in the bag using electronic conductance measurements, which do not provide any specific diagnostic information.

In one aspect, the current disclosure relates to a urine collection bag with an integrated complete infection detection and monitoring system comprising the device of any one of the embodiments disclosed herein. In one aspect, the current disclosure relates to a urine collection bag comprising the device of any one of the embodiments disclosed herein.

In one aspect, the current disclosure relates to a catheter bag comprising the device of any one of the embodiments disclosed herein.

In one aspect, the current disclosure relates to a negative pressure wound therapy system comprising a wound dressing, and a negative pressure source and a wound exudate collection container, wherein wound exudate collection container comprises the device of any one of the embodiments disclosed herein, optionally wherein the dressing comprises oxidized regenerated cellulose (ORC) and/or collagen.

In one aspect, the current disclosure relates to a negative pressure wound therapy with installation system comprising a wound dressing, an instillation fluid, an instillation pump, and a negative pressure source and a wound exudate collection container, wherein wound exudate collection container comprises the device of any one of the embodiments disclosed herein, optionally wherein the dressing comprises oxidized regenerated cellulose (ORC) and/or collagen.

### Methods of Detecting an Infection

In one aspect, the current disclosure relates to a method of detecting an infection in a biological sample of a subject, the method comprising (i) contacting the biological sample from the subject with a device for detecting an infection, wherein the device comprises an electrochemical sensor array; and (ii) measuring a presence, absence or amount of one or more target molecule and/or a metabolic activity within the biological sample, wherein the target molecule and/or the metabolic activity is associated with the infection, wherein the electrochemical sensor array performs the measuring. In embodiments, the biological sample is collected in a collection device. In embodiments, collection device is selected from a catheter bag, a colostomy bag, a urine collection bag, a wound dressing, and a wound exudate collection container. In embodiments, the biological fluid is selected from a body fluid selected from blood, plasma, serum, semen, lacrimal fluid, tears, sputum, saliva, sweat, urine, cerebrospinal fluid, peritoneal fluid, pleural fluid, biopsy sample, feces, lymph, gynecological fluid, skin swab, vaginal swab, oral swab, nasal swab, hair, washing or lavage such as a ductal lavage and broncheoalveolar lavage.

In one aspect, the current disclosure relates to a method of detecting an infection in urine of a subject, the method comprising (i) contacting urine from the subject with a device for detecting an infection, wherein the device comprises an electrochemical sensor array; and (ii) measuring a presence, absence or amount of one or more target molecule and/or a metabolic activity within the urine sample, wherein the target molecule and/or the metabolic activity is associated with the infection, wherein the electrochemical sensor array performs the measuring. In embodiments, the urine is collected in a catheter bag or a urine collection bag. In embodiments, the urinary tract infection is catheter associated urinary tract infections (CAUTI).

In one aspect, the current disclosure relates to a method of detecting a urinary tract infection in a subject, the method comprising (i) contacting urine from the subject with a device for detecting an infection, wherein the device comprises an electrochemical sensor array; and (ii) measuring a presence, absence or amount of one or more target molecule and/or a metabolic activity within the urine sample, wherein the target molecule and/or the metabolic activity is associated with the infection, wherein the electrochemical sensor array performs the measuring. In embodiments, the urine is collected in a catheter bag or a urine collection bag. In embodiments, the urinary tract infection is catheter associated urinary tract infections (CAUTI).

In one aspect, the current disclosure relates to a method of detecting a wound infection in a subject, the method comprising (i) contacting wound exudate from the subject with a device for detecting an infection, wherein the device comprises an electrochemical sensor array; and (ii) measuring a presence, absence or amount of one or more target molecule and/or a metabolic activity within the wound exudate, wherein the target molecule and/or the metabolic activity is associated with the infection, wherein the electrochemical sensor array performs the measuring. In embodiments, the wound exudate is collected in a wound dressing or a wound exudate collection container.

In one aspect, the current disclosure relates to a method of detecting a wound infection in a subject, the method comprising: (i) administering a dressing to a wound, optionally wherein the dressing comprises oxidized regenerated cellulose (ORC) and/or collagen, (ii) applying a negative pressure to the wound, (iii) collecting wound exudate in a wound exudate collection container, (iv) contacting wound exudate from a wound dressing or a wound exudate collection container with a device for detecting an infection, wherein the device comprises an electrochemical sensor array; and (v) measuring a presence, absence or amount of one or more target molecule and/or a metabolic activity within the wound dressing or the wound exudate, wherein the target molecule and/or the metabolic activity is associated with the infection, wherein the electrochemical sensor array performs the measuring.

In one aspect, the current disclosure relates to a method of detecting a gastro-intestinal tract infection in a subject, the method comprising: (i) contacting stool sample from the subject with a device for detecting an infection, wherein the device comprises an electrochemical sensor array; and (ii) measuring a presence, absence or amount of one or more target molecule and/or a metabolic activity within the stool sample, wherein the target molecule and/or the metabolic activity is associated with the infection, wherein the electrochemical sensor array performs the measuring.

In one aspect, the current disclosure relates to a method of detecting a gastro-intestinal tract infection in a subject, the method comprising: (i) contacting biopsy sample from the subject with a device for detecting an infection, wherein the device comprises an electrochemical sensor array; and (ii) measuring a presence, absence or amount of one or more target molecule and/or a metabolic activity within the biopsy sample, wherein the target molecule and/or the metabolic activity is associated with the infection, wherein the electrochemical sensor array performs the measuring. In embodiments, the biopsy sample is obtained using an endoscopic biopsy.

In any of the embodiments disclosed herein the method further comprises estimating a number of viable cells of a pathogen associated with the infection based on the presence, absence or amount of the target molecule and/or the metabolic activity. In embodiments, the method informs the withholding of one or more antibiotics upon a negative test for infection. In embodiments, the method informs the selection of an appropriate antibiotic for the infection upon a positive test for infection. In embodiments, the method further comprises administering an appropriate antibiotic for the infection upon a positive test for infection.

In embodiments, the electrochemical sensor array further comprises a sensor selected from a pH sensor and a temperature sensor. In embodiments, the electrochemical sensor array detects a change in pH, a change in temperature, an electrochemical reaction, binding to an aptamer, a change in color, and the combination of any two or more thereof. In embodiments, each electrochemical sensor of the electrochemical sensor array independently performs an electrochemical measurement. In embodiments, the electrochemical measurement is selected from square wave voltammetry, linear sweep voltammetry, staircase voltammetry, cyclic voltammetry, normal pulse voltammetry, differential pulse voltammetry, and chronoamperometry. In embodiments, the electrochemical measurement is square wave voltammetry and the current flow is measured in response to one or more square wave potentials.

In embodiments, the working electrode is comprised of gold (Au), silver (Ag), platinum (Pt), indium tin oxide (ITO), carbon, carbon nanotubes, carbon nanofibers, graphene, carbon-platinum composites, carbon nanotubes with gold nanoparticles, and any combination thereof. In embodiments, the electrochemical sensor further comprise a reference electrode, optionally wherein the reference electrode is comprised of silver (Ag), silver chloride (AgCl), and platinum (Pt), and any combination thereof.

In embodiments, each electrochemical sensor comprises a second working electrode, wherein the working electrode is one of an oxidizing electrode and a reducing electrode, and the second working electrode is the other of the oxidizing electrode and the reducing electrode. In embodiments, the second working electrode is comprised of gold (Au), silver (Ag), platinum (Pt), indium tin oxide (ITO), carbon, carbon nanotubes, carbon nanofibers, graphene, carbon-platinum composites, carbon nanotubes with gold nanoparticles, and any combination thereof.

In embodiments, the infection is caused by a pathogen. In embodiments, the pathogen is selected from a bacterium and a fungus. In embodiments, the bacterium is selected from Pseudomonas aeruginosa, Proteus mirabilis, Escherichia coli, Klebsiella pneumoniae, other Klebsiella species, Staphylococcus aureus, Enterococcus faecalis, other Enterococcus species, Acinetobacter baumannii, Streptococcus group A species, Streptcoccus group B species, Staphylococcus epidermidis, Clostridium difficile, and Salmonella enterica. In embodiments, the fungus is Candida albicans. In embodiments, the parasite is selected from Giardia, a fecal float worm, fecal roundworm and fecal flatworm.

In embodiments, the device is capable of contemporaneously detecting at least two, or at least three, or at least 4 signals. In embodiments, the signal is associated with a pathogen. In embodiments, the device is capable of contemporaneously detecting the presence or absence of at least two, or at least 3, or at least 4, or at least 5, or at least 6, or at least 7, or at least 8, or at least 9, or at least 10 pathogens.

In embodiments, the device detects a target molecule, and/or a metabolic activity of the pathogen. In embodiments, the target molecule is a quorum sensing molecule. In embodiments, the target molecule is a redox molecule.

In embodiments, the metabolic activity causes breakdown of a basic molecule. In embodiments, the metabolic activity is a urease activity. In embodiments, the metabolic activity changes pH of the urine or the wound exudate. In embodiments, the change in pH is an increase in pH.

In embodiments, the device detects the presence, absence or amount of the target molecule and/or the metabolic activity and/or the metabolic activity of the pathogen. In embodiments, the presence, absence or amount of the target molecule and/or the metabolic activity is measured as current flow through the working electrode. In embodiments, the presence, absence or amount of the target molecule and/or the metabolic activity is measured as current flow through the oxidizing electrode and the reducing electrode. In embodiments, the presence, absence or amount of the target molecule and/or the metabolic activity is measured as a change in pH. In embodiments, the target molecule is selected from quorum sensing molecules (without limitations, e.g., pyocyanin, E. coli autoinducer-2 (AI-2), N-Acyl Homoserine Lactones (AHL)), siderophores (without limitations, e.g. enterobactin, aerobactin, vibriobactin, salmochelin, pyoverdine, and pyochelin), cyclic signaling peptides (without limitations, e.g. Staphylococcus aureus autoinducing peptide (AIP), including AIP variants I to IV, and Enterococcus faecalis gelatinase biosynthesis activating pheromone (GBAP)), and autoinducers (without limitations, e.g. acylated homoserine lactones (AHLs), including N-(3-oxododecanoyI)-homoserine lactone and N-(butyryl)-homoserine lactone, 2-heptyl-3-hydroxy-4-quinolone (PQS), AIP variants Ito IV). In embodiments, the target molecule is selected from 3O-C12-homoserine lactone (3OXO), putrescine, Shiga toxin, aerobactin, auto inducing peptide-1 (AIP-1), gelatinase biosynthesis activating peptide (GBAP), EppR (Protein), short hydrophobic peptide 3 SHP3 (also known as SHP1520), autoinducing peptide (AIP), autoinducing peptide 2 (AIP 2), pyocyanin, enterobactin, tyrosol and farnesol. In embodiments, the presence of the target molecule is indicative of a presence and/or an amount of and/or a number of viable cells of the pathogen. In embodiments, the pathogen is selected from Pseudomonas aeruginosa, Proteus mirabilis, Escherichia coli, Klebsiella pneumoniae, other Klebsiella species, Staphylococcus aureus, Enterococcus faecalis, other Enterococcus species, Acinetobacter baumannii, Streptococcus group A species, Streptcoccus group B species, Staphylococcus epidermidis, Clostridium difficile, Salmonella enterica, Candida albicans, Cryptococcus neoformans, and Aspergillus species.

In embodiments, the presence, absence or amount of: 3O-C12-homoserine lactone (3OXO) is indicative of the presence and/or the amount of and/or the number of viable cells of Pseudomonas aeruginosa, putrescine is indicative of the presence and/or the amount of and/or the number of viable cells of Proteus mirabilis, Shiga toxin is indicative of the presence and/or the amount of and/or the number of viable cells of Escherichia coli, aerobactin is indicative of the presence and/or the amount of and/or the number of viable cells of Klebsiella and/or E. coli, auto inducing peptide-1 (AIP-1) is indicative of the presence and/or the amount of and/or the number of viable cells of Staphylococcus aureus, gelatinase biosynthesis activating peptide (GBAP) is indicative of the presence and/or the amount of and/or the number of viable cells of Enterococcus faecalis, EppR protein is indicative of the presence and/or the amount of and/or the number of viable cells of Acinetobacter baumannii, short hydrophobic peptide 3 SHP3 (SHP1520) is indicative of the presence and/or the amount of and/or the number of viable cells of Streptococcus agalactiae, autoinducing peptide (AIP) is indicative of the presence and/or the amount of and/or the number of viable cells of Staphylococcus pseudintermedius, autoinducing peptide 2 (AIP 2) is indicative of the presence and/or the amount of and/or the number of viable cells of Staphylococcus epidermidis, pyocyanin is indicative of the presence and/or the amount of and/or the number of viable cells of Pseudomonas aeruginosa, tyrosol is indicative of the presence and/or the amount of and/or the number of viable cells of Candida albicans, farnesol is indicative of the presence and/or the amount of and/or the number of viable cells of Candida albicans, and/or enterobactin is indicative of the presence and/or the amount of and/or the number of viable cells of uropathogenic Escherichia coli.

In embodiments, the device is electrically connected or connectable to a reader. In embodiments, the reader provides an output of a presence and/or an amount of and/or a number of viable cells of a pathogen. In embodiments, the reader is capable of transmitting the specific signals to a display device. In embodiments, the signal is wirelessly transmitted. In embodiments, the pathogen is selected from Pseudomonas aeruginosa, Proteus mirabilis, Escherichia coli, Klebsiella pneumonia, other Klebsiella species, Staphylococcus aureus, Enterococcus faecalis, other Enterococcus species, Acinetobacter baumannii, Streptococcus group A species, Streptococcus Group B species, Staphylococcus epidermidis, Pseudomonas aeruginosa, Clostridium difficile, and Salmonella enterica, Borrelia burgdorferi, Candida albicans, Cryptococcus neoformans, and Aspergillus species.

In embodiments, the device detects the presence and/or the amount of and/or the number of viable cells of a pathogen in less than one hour, less than 45 minutes, or less than 30 minutes, or less than 15 minutes, or less than 10 minutes, or less than 5 minutes, or less than 2 minutes or less than 1 minute.

### Methods of Selecting a Patient for Therapy

In one aspect, the current disclosure relates to a method of selecting a catheterized patient having or suspected as having a urinary tract infection for therapy, the method comprising: (i) contacting urine from a catheter bag or urine collection bag collected from a subject with a device of any one of the embodiments disclosed herein for detecting an infection, wherein the device comprises an electrochemical sensor array; (ii) measuring a presence, absence or amount of one or more target molecule and/or a metabolic activity within the urine sample, wherein the target molecule and/or the metabolic activity is associated with the infection; and (iii) selecting the patient for therapy with an appropriate antibiotic for the infection upon a positive test for infection.

In one aspect, the current disclosure relates to a method of selecting a patient having or suspected as having a wound infection for therapy, the method comprising: (i) contacting wound exudate from a wound dressing or a wound exudate collection container with a device of any one of the embodiments disclosed herein for detecting an infection, wherein the device comprises an electrochemical sensor array; (ii) measuring a presence, absence or amount of one or more target molecule and/or a metabolic activity within the wound dressing or the wound exudate, wherein the target molecule and/or the metabolic activity is associated with the infection; and (iii) selecting the patient for therapy with an appropriate antibiotic for the infection upon a positive test for infection.

In one aspect, the current disclosure relates to a method of selecting a patient having or suspected as having a wound infection for therapy, the method comprising: (i) administering a dressing to a wound, optionally wherein the dressing comprises oxidized regenerated cellulose (ORC) and/or collagen, (ii) applying a negative pressure to the wound, (iii) collecting wound exudate in a wound exudate collection container, (iv) contacting wound exudate from a wound dressing or a wound exudate collection container with a device of any one of the embodiments disclosed herein for detecting an infection, wherein the device comprises an electrochemical sensor array; (v) measuring a presence, absence or amount of one or more target molecule and/or a metabolic activity within the wound exudate, wherein the target molecule and/or the metabolic activity is associated with the infection; and (vi) selecting the patient for therapy with an appropriate antibiotic for the infection upon a positive test for infection.

In one aspect, the current disclosure relates to a method for preventing a catheter-associated bacteremia or sepsis in a catheterized patient, the method comprising (i) contacting urine from the subject with a device of any one of the embodiments disclosed herein for detecting an infection, wherein the device comprises an electrochemical sensor array; (ii) measuring an amount of one or more target molecule and/or a metabolic activity within the urine sample, wherein the target molecule and/or the metabolic activity is associated with the infection; (iii) comparing the amount of the target molecule and/or the metabolic activity with the amount of the target molecule and/or the metabolic activity prior to therapy, from another healthy subject, or a standard and thereby detecting the presence of an infection; and (iv) administering therapy of an appropriate antibiotic for the infection.

### Methods of Determining Efficacy of Therapy

In one aspect, the current disclosure relates to a method for determining efficacy of a therapy in a catheterized patient receiving the therapy for a urinary tract infection, the method comprising (i) contacting urine from the subject receiving the therapy with a device of any one of the embodiments disclosed herein for detecting an infection, wherein the device comprises an electrochemical sensor array; (ii) measuring an amount of one or more target molecule and/or a metabolic activity within the urine sample, wherein the target molecule and/or the metabolic activity is associated with the infection; and (iii) comparing the amount of the target molecule and/or the metabolic activity with the amount of the target molecule and/or the metabolic activity prior to therapy, from another healthy subject, or a standard.

In one aspect, the current disclosure relates to a method for determining efficacy of a therapy in a patient receiving the therapy for a wound infection, the method comprising (i) contacting wound exudate from the subject receiving the therapy with a device of any one of the embodiments disclosed herein for detecting an infection, wherein the device comprises an electrochemical sensor array; (ii) measuring a presence, absence or amount of one or more target molecule and/or a metabolic activity within the wound exudate, wherein the target molecule and/or the metabolic activity is associated with the infection; and (iii) comparing the amount of the target molecule and/or the metabolic activity with the amount of the target molecule and/or the metabolic activity prior to therapy, from another healthy subject, or a standard.

### Subjects

In embodiments, the subject is a human. In embodiments, the subject is catheterized. In embodiments, the subject resides in a long term care facility. In embodiments, the subject is suffering from or is at risk of suffering from a urinary tract infection. In embodiments, the subject is suffering from a wound. In embodiments, the wound is a chronic wound. In embodiments, the wound is a diabetic wound. In embodiments, the wound is an acute wound. In embodiments, the subject is undergoing a wound therapy with a wound dressing. In embodiments, the subject is undergoing a negative pressure wound therapy. In embodiments, the subject is undergoing a negative pressure wound therapy with instillation.

### SELECT EMBODIMENTS

Embodiment 1. A method of detecting an infection in urine of a subject, the method comprising
(i) contacting urine from the subject with a device for detecting an infection, wherein the device comprises an electrochemical sensor array; and
(ii) measuring a presence, absence or amount of one or more target molecule and/or a metabolic activity within the urine sample, wherein the target molecule and/or the metabolic activity is associated with the infection, wherein the electrochemical sensor array performs the measuring.

Embodiment 2. A method of detecting a urinary tract infection in a subject, the method comprising
(i) contacting urine from the subject with a device for detecting an infection, wherein the device comprises an electrochemical sensor array; and
(ii) measuring a presence, absence or amount of one or more target molecule and/or a metabolic activity within the urine sample, wherein the target molecule and/or the metabolic activity is associated with the infection, wherein the electrochemical sensor array performs the measuring.

Embodiment 3. The method of Embodiment 1 or Embodiment 2, wherein the urine is collected in a catheter bag or a urine collection bag.

Embodiment 4. The method of any one of Embodiments 1 to 3, wherein the urinary tract infection is catheter associated urinary tract infections (CAUTI).

Embodiment 5. A method of detecting a wound infection in a subject, the method comprising
(i) contacting wound exudate from the subject with a device for detecting an infection, wherein the device comprises an electrochemical sensor array; and
(ii) measuring a presence, absence or amount of one or more target molecule and/or a metabolic activity within the wound exudate, wherein the target molecule and/or the metabolic activity is associated with the infection, wherein the electrochemical sensor array performs the measuring.

Embodiment 6. The method of Embodiment 5, wherein the wound exudate is collected in a wound dressing or a wound exudate collection container.

Embodiment 7. A method of detecting a wound infection in a subject, the method comprising:
(i) administering a dressing to a wound, optionally wherein the dressing comprises oxidized regenerated cellulose (ORC) and/or collagen,
(ii) applying a negative pressure to the wound,
(iii) collecting wound exudate in a wound exudate collection container,
(iv) contacting wound exudate from a wound dressing or a wound exudate collection container with a device for detecting an infection, wherein the device comprises an electrochemical sensor array; and
(v) measuring a presence, absence or amount of one or more target molecule and/or a metabolic activity within the wound exudate, wherein the target molecule and/or the metabolic activity is associated with the infection, wherein the electrochemical sensor array performs the measuring.

Embodiment 8. The method of any one of Embodiments 1 to 7, further comprising estimating a number of viable cells of a pathogen associated with the infection based on the presence, absence or amount of the target molecule and/or the metabolic activity.

Embodiment 9. The method of any one of Embodiments 1 to 8, wherein the method informs the withholding of one or more antibiotics upon a negative test for infection.

Embodiment 10. The method of any one of Embodiments 1 to 9, wherein the method informs the selection of an appropriate antibiotic for the infection upon a positive test for infection.

Embodiment 11. The method of Embodiment 10, further comprising administering an appropriate antibiotic for the infection upon a positive test for infection.

Embodiment 12. The method of any one of Embodiments 1 to 11, wherein the electrochemical sensor array further comprises a sensor selected from a pH sensor and a temperature sensor.

Embodiment 13. The method of any one of Embodiments 1 to 12, wherein the electrochemical sensor array detects a change in pH, a change in temperature, an electrochemical reaction, binding to an aptamer, a change in color, and the combination of any two or more thereof.

Embodiment 14. The method of any one of Embodiments 1 to 13, wherein each electrochemical sensor of the electrochemical sensor array independently performs an electrochemical measurement.

Embodiment 15. The method of Embodiment 14, wherein the electrochemical measurement is selected from square wave voltammetry, linear sweep voltammetry, staircase voltammetry, cyclic voltammetry, normal pulse voltammetry, differential pulse voltammetry, and chronoamperometry.

Embodiment 16. The method of Embodiment 14 or Embodiment 15, wherein the electrochemical measurement is square wave voltammetry and the current flow is measured in response to one or more square wave potentials.

Embodiment 17. The method of any one of Embodiments 1 to 16, wherein each electrochemical sensor comprises a second working electrode, wherein the working electrode is one of an oxidizing electrode and a reducing electrode, and the second working electrode is the other of the oxidizing electrode and the reducing electrode.

Embodiment 18. The method of Embodiment 17, wherein the working electrode is comprised of gold (Au), silver (Ag), platinum (Pt), indium tin oxide (ITO), carbon, carbon nanotubes, carbon nanofibers, graphene, carbon-platinum composites, carbon nanotubes with gold nanoparticles, and any combination thereof.

Embodiment 19. The method of Embodiment 17 or Embodiment 18, wherein the electrochemical sensor further comprise a reference electrode, optionally wherein the reference electrode is comprised of silver (Ag), silver chloride (AgCl), and platinum (Pt), and any combination thereof.

Embodiment 20. The method of any one of Embodiments 1 to 19, wherein the infection is caused by a pathogen.

Embodiment 21. The method of Embodiment 20, wherein the pathogen is selected from a bacterium and a fungus.

Embodiment 22. The method of Embodiment 21, wherein the bacterium is selected from Pseudomonas aeruginosa, Proteus mirabilis, Escherichia coli, Klebsiella pneumoniae, other Klebsiella species, Staphylococcus aureus, Enterococcus faecalis, other Enterococcus species, Acinetobacter baumannii, Streptococcus group A species, Streptococcus Group B species, Staphylococcus epidermidis, Pseudomonas aeruginosa, Clostridium difficile, and Salmonella enterica.

Embodiment 23. The method of Embodiment 21, wherein the fungus is selected from Candida albicans, Cryptococcus neoformans, and Aspergillus species.

Embodiment 24. The method of Embodiment 23, wherein the fungus is Candida albicans.

Embodiment 25. The method of any one of Embodiments 1 to 24, wherein the device is capable of contemporaneously detecting the presence or absence of at least two, or at least 3, or at least 4, or at least 5, or at least 6, or at least 7, or at least 8, or at least 9, or at least 10 pathogens.

Embodiment 26. The method of any one of Embodiments 1 to 25, wherein the device is capable of contemporaneously detecting at least two, or at least three, or at least 4 signals.

Embodiment 27. The method of Embodiment 26, wherein the signal is associated with the pathogen.

Embodiment 28. The method of any one of Embodiments 1 to 27, wherein the device detects a target molecule, and/or a metabolic activity of the pathogen.

Embodiment 29. The method of Embodiment 28, wherein the target molecule is a quorum sensing molecule.

Embodiment 30. The method of Embodiment 28 or Embodiment 29, wherein the target molecule is a redox molecule.

Embodiment 31. The method of Embodiment 28, wherein the metabolic activity causes breakdown of a basic molecule.

Embodiment 32. The method of Embodiment 31, wherein the metabolic activity is a urease activity.

Embodiment 33. The method of Embodiment 31 or Embodiment 32, wherein the metabolic activity changes pH of the urine or the wound exudate.

Embodiment 34. The method of Embodiment 33, wherein the change in pH is an increase in pH.

Embodiment 35. The method of any one of Embodiments 28 to 34, wherein the device detects the presence, absence or amount of the target molecule and/or the metabolic activity and/or the metabolic activity of the pathogen.

Embodiment 36. The method of Embodiment 35, wherein the presence, absence or amount of the target molecule and/or the metabolic activity is measured as current flow through the working electrode.

Embodiment 37. The method of Embodiment 35 or Embodiment 36, wherein the presence, absence or amount of the target molecule and/or the metabolic activity is measured as current flow through the oxidizing electrode and the reducing electrode.

Embodiment 38. The method of Embodiment 35 or Embodiment 36, wherein the presence, absence or amount of the target molecule and/or the metabolic activity is measured as a change in pH.

Embodiment 39. The method of any one of Embodiments 28 to 30 or 35-38, wherein the target molecule is selected from 3O-C12-homoserine lactone (3OXO), putrescine, Shiga toxin, aerobactin, auto inducing peptide-1 (AIP-1), gelatinase biosynthesis activating peptide (GBAP), EppR (Protein), short hydrophobic peptide 3 SHP3 (also known as SHP1520), autoinducing peptide (AIP), autoinducing peptide 2 (AIP 2), pyocyanin, enterobactin, tyrosol and farnesol.

Embodiment 40. The method of Embodiment 39, wherein the presence of the target molecule is indicative of a presence and/or an amount of and/or a number of viable cells of the pathogen.

Embodiment 41. The method of Embodiment 39 or Embodiment 40, wherein the pathogen is selected from Pseudomonas aeruginosa, Proteus mirabilis, Escherichia coli, Klebsiella pneumoniae, other Klebsiella species, Staphylococcus aureus, Enterococcus faecalis, other Enterococcus species, Acinetobacter baumannii, Streptococcus group A species, Streptococcus Group B species, Staphylococcus epidermidis, Pseudomonas aeruginosa, Clostridium difficile, Salmonella enterica, Candida albicans, Cryptococcus neoformans, and Aspergillus species.

Embodiment 42. The method of Embodiment 40, wherein the presence, absence or amount of:
3O-C12-homoserine lactone (3OXO) is indicative of the presence and/or the amount of and/or the number of viable cells of Pseudomonas aeruginosa,
putrescine is indicative of the presence and/or the amount of and/or the number of viable cells of Proteus mirabilis,
Shiga toxin is indicative of the presence and/or the amount of and/or the number of viable cells of Escherichia coli,
aerobactin is indicative of the presence and/or the amount of and/or the number of viable cells of Klebsiella and/or E. coli,
auto inducing peptide-1 (AIP-1) is indicative of the presence and/or the amount of and/or the number of viable cells of Staphylococcus aureus,
gelatinase biosynthesis activating peptide (GBAP) is indicative of the presence and/or the amount of and/or the number of viable cells of Enterococcus faecalis,
EppR protein is indicative of the presence and/or the amount of and/or the number of viable cells of Acinetobacter baumannii,
short hydrophobic peptide 3 SHP3 (SHP1520) is indicative of the presence and/or the amount of and/or the number of viable cells of Streptococcus agalactiae,
autoinducing peptide (AIR) is indicative of the presence and/or the amount of and/or the number of viable cells of Staphylococcus pseudintermedius,
autoinducing peptide 2 (AIP 2) is indicative of the presence and/or the amount of and/or the number of viable cells of Staphylococcus epidermidis,
pyocyanin is indicative of the presence and/or the amount of and/or the number of viable cells of Pseudomonas aeruginosa,
tyrosol is indicative of the presence and/or the amount of and/or the number of viable cells of Candida albicans,
farnesol is indicative of the presence and/or the amount of and/or the number of viable cells of Candida albicans, and/or
enterobactin is indicative of the presence and/or the amount of and/or the number of viable cells of uropathogenic Escherichia coli.

Embodiment 43. The method of any one of Embodiments 1 to 42, wherein the device is electrically connected or connectable to a reader.

Embodiment 44. The method of Embodiment 43, wherein the reader provides an output of a presence and/or an amount of and/or a number of viable cells of a pathogen.

Embodiment 45. The method of Embodiment 43, wherein the pathogen is selected from a bacterium and a fungus.

Embodiment 46. The method of Embodiment 44, wherein the pathogen is selected from Pseudomonas aeruginosa, Proteus mirabilis, Escherichia coli, Klebsiella pneumoniae, Staphylococcus aureus, Enterococcus faecalis, Acinetobacter baumannii, Streptococcus Group A species, Streptococcus Group B species, Staphylococcus epidermidis, Pseudomonas aeruginosa, Candida albicans, Cryptococcus neoformans, and Aspergillus species.

Embodiment 47. The method of Embodiment 45 or 46, wherein the device detects the presence and/or the amount of and/or the number of viable cells of a pathogen in less than one hour, less than 45 minutes, or less than 30 minutes, or less than 15 minutes, or less than 10 minutes, or less than 5 minutes, or less than 2 minutes or less than 1 minute.

Embodiment 48. The method of any one of Embodiments 1 to 44, wherein the device detects the infection in less than one hour, less than 45 minutes, or less than 30 minutes, or less than 15 minutes, or less than 10 minutes, or less than 5 minutes, or less than 2 minutes or less than 1 minute.

Embodiment 49. A device for detecting an infection in a subject comprising an electrochemical sensor array, wherein electrochemical sensor array comprises a plurality of electrochemical sensors, wherein each electrochemical sensor comprises a working electrode, a reference electrode, and a counter electrode, wherein the electrochemical sensor array is fluidically connected to a wound exudate in a wound dressing or a wound exudate collection container, a wound exudate collection container of negative pressure wound therapy, a fluid collection container of negative pressure wound therapy with instillation, or urine in a catheter bag or a urine collection bag.

Embodiment 50. The device of Embodiment 49, wherein the electrochemical sensor array further comprises a sensor selected from a pH sensor and a temperature sensor.

Embodiment 51. The device of Embodiment 49 or Embodiment 50, wherein the electrochemical sensor array detects a change in pH, a change in temperature, an electrochemical reaction, binding to an aptamer, a change in color, or the combination of any two or more thereof.

Embodiment 52. The device of any one of Embodiments 49 to 51, wherein the device is capable of contemporaneously detecting at least two, or at least three, or at least 4 signals.

Embodiment 53. The device of Embodiment 52, wherein the signal is associated with a pathogen.

Embodiment 54. The device of any one of Embodiments 49 to 53, wherein each electrochemical sensor is capable of independently performing an electrochemical measurement.

Embodiment 55. The device of Embodiment 52, wherein the electrochemical measurement is selected from square wave voltammetry, linear sweep voltammetry, staircase voltammetry, cyclic voltammetry, normal pulse voltammetry, differential pulse voltammetry, and chronoamperometry.

Embodiment 56. The device of Embodiment 53 or Embodiment 54, wherein the electrochemical measurement is square wave voltammetry.

Embodiment 57. The device of any one of Embodiments 49 to 56, wherein the electrochemical measurement is measurement of a current flow.

Embodiment 58. The device of Embodiment 57, wherein the current flow is measured in response to one or more square wave potentials.

Embodiment 59. The device of any one of Embodiments 49 to 57, wherein the working electrode is comprised of gold (Au), silver (Ag), platinum (Pt), indium tin oxide (ITO), carbon, carbon nanotubes, carbon nanofibers, graphene, carbon-platinum composites, carbon nanotubes with gold nanoparticles, or any combination thereof.

Embodiment 60. The device of any one of Embodiments 49 to 59, wherein the electrochemical sensors further comprise a reference electrode, optionally wherein the reference electrode is comprised of silver (Ag), silver chloride (AgCl), and platinum (Pt), and any combination thereof.

Embodiment 61. The device of any one of Embodiments 49 to 60, wherein each electrochemical sensor comprises a second working electrode.

Embodiment 62. The device of Embodiment 61, wherein the second working electrode is comprised of gold (Au), silver (Ag), platinum (Pt), indium tin oxide (ITO), carbon, carbon nanotubes, carbon nanofibers, graphene, carbon-platinum composites, carbon nanotubes with gold nanoparticles, or any combination thereof.

Embodiment 63. The device of Embodiment 61 or Embodiment 62, wherein the working electrode is one of an oxidizing electrode and a reducing electrode, and the second working electrode is the other of the oxidizing electrode and the reducing electrode.

Embodiment 64. The device of any one of Embodiments 49 to 63, wherein the device is capable of detecting an infection caused by a pathogen.

Embodiment 65. The device of Embodiment 53 or 64, wherein the pathogen is selected a bacterium, a fungus and a parasite.

Embodiment 66. The device of Embodiment 65, wherein the bacterium is selected from Pseudomonas aeruginosa, Proteus mirabilis, Escherichia coli, Klebsiella pneumoniae, other Klebsiella species, Staphylococcus aureus, Enterococcus faecalis, other Enterococcus species, Acinetobacter baumannii, Streptococcus group A species, Streptcoccus group B species, Staphylococcus epidermidis, Pseudomonas aeruginosa, Clostridium difficile, and Salmonella enterica.

Embodiment 67. The device of Embodiment 65, wherein the fungus is Candida albicans.

Embodiment 68. The device of Embodiment 65, wherein the parasite is Giardia, a fecal float worm, fecal roundworm and fecal flatworm.

Embodiment 69. The device of any one of Embodiments 49 to 68, wherein the electrochemical sensor array is capable of contemporaneously detecting the presence or absence of at least two, or at least 3, or at least 4, or at least 5, or at least 6, or at least 7, or at least 8, or at least 9, or at least 10 pathogens.

Embodiment 70. The device of any one of Embodiments 49 to 69, wherein the device detects a target molecule, and/or a metabolic activity of the pathogen.

Embodiment 71. The device of Embodiment 70, wherein the target molecule is a quorum sensing molecule.

Embodiment 72. The device of Embodiment 70 or Embodiment 72, wherein the target molecule is a redox molecule.

Embodiment 73. The device of Embodiment 70, wherein the metabolic activity causes breakdown of a basic molecule.

Embodiment 74. The device of Embodiment 73, wherein the metabolic activity is a urease activity.

Embodiment 75. The device of Embodiment 73 or Embodiment 74, wherein the metabolic activity changes pH of the urine or the wound exudate.

Embodiment 76. The device of Embodiment 75, wherein the change in pH is an increase in pH.

Embodiment 77. The device of any one of Embodiments 70 to 76, wherein the device detects the presence, absence or amount of the target molecule and/or the metabolic activity and/or the metabolic activity of the pathogen.

Embodiment 78. The device of Embodiment 77, wherein the presence, absence or amount of the target molecule and/or the metabolic activity is measured as current flow through the working electrode.

Embodiment 79. The device of Embodiment 77 or Embodiment 78, wherein the presence, absence or amount of the target molecule and/or the metabolic activity is measured as current flow through the oxidizing electrode and the reducing electrode.

Embodiment 80. The device of Embodiment 77 or Embodiment 78, wherein the presence, absence or amount of the target molecule and/or the metabolic activity is measured as a change in pH.

Embodiment 81. The device of any one of Embodiments 70 to 72 or 77 to 80, wherein the target molecule is selected from 3O-C12-homoserine lactone (3OXO), putrescine, Shiga toxin, aerobactin, auto inducing peptide-1 (AIP-1), gelatinase biosynthesis activating peptide (GBAP), EppR (Protein), short hydrophobic peptide 3 SHP3 (also known as SHP1520), autoinducing peptide (AIP), autoinducing peptide 2 (AIP 2), pyocyanin, enterobactin, tyrosol and farnesol.

Embodiment 82. The device of Embodiment 81, wherein the presence of the target molecule is indicative of a presence and/or an amount of and/or a number of viable cells of the pathogen.

Embodiment 83. The device of Embodiment 81 or Embodiment 82, wherein the pathogen is selected from Pseudomonas aeruginosa, Proteus mirabilis, Escherichia coli, Klebsiella pneumoniae, other Klebsiella species, Staphylococcus aureus, Enterococcus faecalis, other Enterococcus species, Acinetobacter baumannii, Streptococcus group A species, Streptococcus group B species, Staphylococcus epidermidis, Pseudomonas aeruginosa, Clostridium difficile, Salmonella enterica, Candida albicans, Cryptococcus neoformans, and Aspergillus species.

Embodiment 84. The device of Embodiment 83, wherein the presence, absence or amount of:
3O-C12-homoserine lactone (3OXO) is indicative of the presence and/or the amount of and/or the number of viable cells of Pseudomonas aeruginosa,
putrescine is indicative of the presence and/or the amount of and/or the number of viable cells of Proteus mirabilis,
Shiga toxin is indicative of the presence and/or the amount of and/or the number of viable cells of Escherichia coli,
aerobactin is indicative of the presence and/or the amount of and/or the number of viable cells of Klebsiella and/or E. coli,
auto inducing peptide-1 (AIP-1) is indicative of the presence and/or the amount of and/or the number of viable cells of Staphylococcus aureus,
gelatinase biosynthesis activating peptide (GBAP) is indicative of the presence and/or the amount of and/or the number of viable cells of Enterococcus faecalis,
EppR protein is indicative of the presence and/or the amount of and/or the number of viable cells of Acinetobacter baumannii,
short hydrophobic peptide 3 SHP3 (SHP1520) is indicative of the presence and/or the amount of and/or the number of viable cells of Streptococcus agalactiae,
autoinducing peptide (AIP) is indicative of the presence and/or the amount of and/or the number of viable cells of Staphylococcus pseudintermedius,
autoinducing peptide 2 (AIP 2) is indicative of the presence and/or the amount of and/or the number of viable cells of Staphylococcus epidermidis,
pyocyanin is indicative of the presence and/or the amount of and/or the number of viable cells of Pseudomonas aeruginosa,
tyrosol is indicative of the presence and/or the amount of and/or the number of viable cells of Candida albicans,
farnesol is indicative of the presence and/or the amount of and/or the number of viable cells of Candida albicans, and/or
enterobactin is indicative of the presence and/or the amount of and/or the number of viable cells of uropathogenic Escherichia coli.

Embodiment 85. The device of any one of Embodiments 49 to 84, wherein the device is electrically connected or connectable to a reader.

Embodiment 86. The device of Embodiment 85, wherein the reader provides an output of a presence and/or an amount of and/or a number of viable cells of a pathogen.

Embodiment 87. The device of Embodiment 86, wherein the pathogen is selected from a bacterium and a fungus.

Embodiment 88. The device of Embodiment 86, wherein the pathogen is selected from Pseudomonas aeruginosa, Proteus mirabilis, Escherichia coli, Klebsiella pneumoniae, other Klebsiella species, Staphylococcus aureus, Enterococcus faecalis, other Enterococcus species, Acinetobacter baumannii, Streptococcus group A species, Streptcoccus group B species, Staphylococcus epidermidis, Pseudomonas aeruginosa, Clostridium difficile, Salmonella enterica, Candida albicans, Cryptococcus neoformans, and Aspergillus species.

Embodiment 89. The device of Embodiment 85 to Embodiment 86, wherein the reader is capable of transmitting the specific signals to a display device.

Embodiment 90. The device of Embodiment 89, wherein the signal is wirelessly transmitted.

Embodiment 91. The device of Embodiment 86, wherein the pathogen is selected from Pseudomonas aeruginosa, Proteus mirabilis, Escherichia coli, Klebsiella pneumoniae, other Klebsiella species, Staphylococcus aureus, Enterococcus faecalis, other Enterococcus species, Acinetobacter baumannii, Streptococcus group A species, Streptcoccus group B species, Staphylococcus epidermidis, Pseudomonas aeruginosa, Clostridium difficile, Salmonella enterica, Candida albicans, Cryptococcus neoformans, and Aspergillus species.

Embodiment 92. The device of any one of Embodiments 49 to 91, wherein the device detects the infection in less than one hour, less than 45 minutes, or less than 30 minutes, or less than 15 minutes, or less than 10 minutes, or less than 5 minutes, or less than 2 minutes or less than 1 minute.

Embodiment 93. A dressing comprising the device of any one of Embodiments 49 to 92, optionally wherein the dressing further comprises oxidized regenerated cellulose (ORC) and/or collagen.

Embodiment 94. A urine collection bag comprising the device of any one of Embodiments 49 to 92.

Embodiment 95. A catheter bag comprising the device of any one of Embodiments 49 to 92.

Embodiment 96. A negative pressure wound therapy system comprising a wound dressing, and a negative pressure source and a wound exudate collection container, wherein wound exudate collection container comprises the device of any one of Embodiments 49 to 92, optionally wherein the dressing comprises oxidized regenerated cellulose (ORC) and/or collagen.

Embodiment 97. A negative pressure wound therapy with installation system comprising a wound dressing, an instillation fluid, an instillation pump, and a negative pressure source and a wound exudate collection container, wherein wound exudate collection container comprises the device of any one of Embodiments 49 to 92, optionally wherein the dressing comprises oxidized regenerated cellulose (ORC) and/or collagen.

Embodiment 98. A method of selecting a catheterized patient having or suspected as having a urinary tract infection for therapy, the method comprising:
(i) contacting urine from a catheter bag or urine collection bag collected from a subject with a device for detecting an infection, wherein the device comprises an electrochemical sensor array;
(ii) measuring a presence, absence or amount of one or more target molecule and/or a metabolic activity within the urine sample, wherein the target molecule and/or the metabolic activity is associated with the infection; and
(iii) selecting the patient for therapy with an appropriate antibiotic for the infection upon a positive test for infection.

Embodiment 99. A method of selecting a patient having or suspected as having a wound infection for therapy, the method comprising:
(i) contacting wound exudate from a wound dressing or a wound exudate collection container with a device for detecting an infection, wherein the device comprises an electrochemical sensor array;
(ii) measuring a presence, absence or amount of one or more target molecule and/or a metabolic activity within the wound dressing or the wound exudate, wherein the target molecule and/or the metabolic activity is associated with the infection; and
(iii) selecting the patient for therapy with an appropriate antibiotic for the infection upon a positive test for infection.

Embodiment 100. A method of selecting a patient having or suspected as having a wound infection for therapy, the method comprising:
(i) administering a dressing to a wound, optionally wherein the dressing comprises oxidized regenerated cellulose (ORC) and/or collagen,
(ii) applying a negative pressure to the wound,
(iii) collecting wound exudate in a wound exudate collection container,
(iv) contacting wound exudate from a wound dressing or a wound exudate collection container with a device for detecting an infection, wherein the device comprises an electrochemical sensor array;
(v) measuring a presence, absence or amount of one or more target molecule and/or a metabolic activity within the wound exudate, wherein the target molecule and/or the metabolic activity is associated with the infection; and
(vi) selecting the patient for therapy with an appropriate antibiotic for the infection upon a positive test for infection.

Embodiment 101. A method for determining efficacy of a therapy in a catheterized patient receiving the therapy for a urinary tract infection, the method comprising
(i) contacting urine from the subject receiving the therapy with a device for detecting an infection, wherein the device comprises an electrochemical sensor array;
(ii) measuring an amount of one or more target molecule and/or a metabolic activity within the urine sample, wherein the target molecule and/or the metabolic activity is associated with the infection; and
(iii) comparing the amount of the target molecule and/or the metabolic activity with the amount of the target molecule and/or the metabolic activity prior to therapy, from another healthy subject, or a standard.

Embodiment 102. A method for preventing a catheter-associated bacteremia or sepsis in a catheterized patient, the method comprising
(i) contacting urine from the subject with a device for detecting an infection, wherein the device comprises an electrochemical sensor array;
(ii) measuring an amount of one or more target molecule and/or a metabolic activity within the urine sample, wherein the target molecule and/or the metabolic activity is associated with the infection;
(iii) comparing the amount of the target molecule and/or the metabolic activity with the amount of the target molecule and/or the metabolic activity prior to therapy, from another healthy subject, or a standard and thereby detecting the presence of an infection; and
(iv) administering therapy of an appropriate antibiotic for the infection.

Embodiment 103. A method for determining efficacy of a therapy in a patient receiving the therapy for a wound infection, the method comprising
(i) contacting wound exudate from the subject receiving the therapy with a device for detecting an infection, wherein the device comprises an electrochemical sensor array;
(ii) measuring a presence, absence or amount of one or more target molecule and/or a metabolic activity within the wound exudate, wherein the target molecule and/or the metabolic activity is associated with the infection; and
(iii) comparing the amount of the target molecule and/or the metabolic activity with the amount of the target molecule and/or the metabolic activity prior to therapy, from another healthy subject, or a standard.

### EXAMPLES

The examples herein are provided to illustrate advantages and benefits of the present technology and to further assist a person of ordinary skill in the art with using the materials, methods and preparing the kits of the present disclosure. The examples herein are also presented in order to more fully illustrate the preferred aspects of the present disclosure. The examples should in no way be construed as limiting the scope of the present disclosure, as defined by the appended claims. The examples can include or incorporate any of the variations, aspects or embodiments of the present technology described above. The variations, aspects or embodiments described above may also further each include or incorporate the variations of any or all other variations, aspects or embodiments of the present disclosure.

### Example 1. Development of E. Coli Aptamer-Based Sensors and Correlation Signal with Infection Level

Enterobactin (without iron, EMC Microcollections), Shiga toxin (B-subunit, Sigma-Aldrich), and aptamers that have already been screened for high affinity binding to the two E. coli target molecules, enterobactin and Shiga toxin were obtained from a vendor. 5 aptamers predicted to bind specifically to enterobactin and 5 aptamers predicted to specifically bind to Shiga toxin were identified based on a library screening procedure.

The binding kinetics of the aptamer/target combination is determined using surface plasmon resonance (SPR). This test provides a K_{d} value which is indicative of the metabolite detection range. The aptamers capable to detect 1 μM for enterobactin and Shiga toxin is selected since 1 μM for enterobactin and Shiga toxin has been reported to be a typical level of metabolite concentration in urinary tract infections. Each test is performed in triplicate and with 3 aptamers running in parallel using a Biacore T200 (GE Healthcare).

The aptamers with the best binding kinetics are then used in electrochemical tests with enterobactin and Shiga toxin spiked phosphate buffered saline solutions (PBS). Multiple variables are optimized to increase the signal-to-noise ratio and total signal change including (1) the immobilized aptamer density and (2) the electrochemical scan frequency.

### Example 2. Aptamer-Based Detection of E. Coli Shiga Toxin

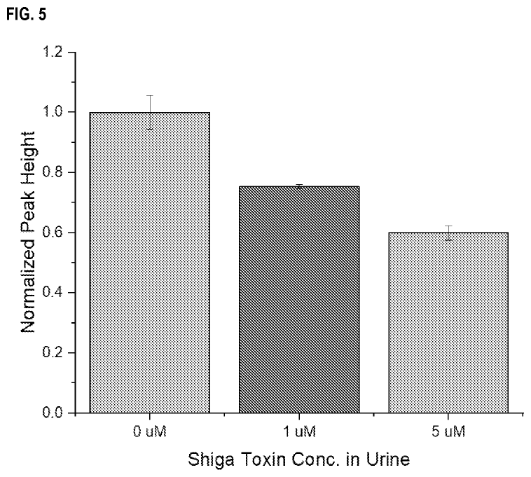

Aptamer immobilization onto the gold electrodes was performed with established thiol-gold binding chemistry along with other proprietary functionalization steps at concentrations of 0.2, 1, and 2 μM. These modified electrodes were then be electrochemically scanned using a square-wave voltammetry technique. The square-wave scans were performed in 50 Hz increments from 50-500 Hz in blank PBS solutions, and compared with PBS solutions containing either 1 μM enterobactin or Shiga toxin. Each of these scans are performed in triplicate with 3 sets of separately modified electrodes. As shown in FIG. 5, the peak current in a Shiga toxin-specific aptamer-based sensor in the presence of increasing concentrations of Shiga toxin. As shown, Shiga toxin aptamer-based electrochemical sensor can detect 1 μM levels of Shiga toxin in urine samples (FIG. 5).

These results demonstrate that the electrochemical sensors disclosed here are capable of detecting Shiga toxin, and thereby detect E. coli. These results also demonstrate that testing the sensors in urine samples does not indicate biofouling to be a significant issue.

### Example 3. Optimization of Aptamer-Based Detection of E. Coli Shiga Toxin

The optimized frequency and surface concentration of aptamer is determined, and the sensors are tested in urine samples obtained from BiolVT (New York) spiked with known concentrations of enterobactin or Shiga toxin. These tests determine the linear range for enterobactin and Shiga toxin detection and form a calibration standard curve.

Finally, ATCC strains of E. coli and STEC are purchased and grown in clean urine (BiolVT) in culture tubes at 37° C., where they will colonize and produce their infection associated metabolites. The sensor's electrochemical signal will then be compared to the calibration standard curve and compared to the bacterial load based on hemocytometer measurements in order to correlate the level of metabolite production to the infection level in CFU/mL.

Multiple aptamers are screened and the best aptamer is chosen. Aptamer libraries from a number of different companies including SomaLogic (Colorado) and Base Pair Biotechnologies (Texas) are screened. If biofoulants, in urine, if any, affect the monitoring capabilities, a filter membrane is integrated over the electrode.

### Example 4. Development of Electrochemical pH Sensor for Proteus and Correlation Signal with Infection Level

Proteus is detected based on a pH increase in the urine due to the production of urease. The electrochemical pH sensor will consist of a short tethered DNA sequence with a redox active methylene blue end group that is redox active.

The redox peak is present at a given potential in known pH solution relative to a stable reference electrode. For example, at pH 7 with a Ag/AgCl reference electrode the peak from methylene blue occurs around −0.3 V when performing a square-wave voltammetry scan.

The pH of purchased urine samples are manually varied in increments of 0.5 pH unit from pH 3-11 using small amounts of HCl or NaOH to make the solution more acidic or basic. These samples are authenticated with a pH probe and then used to calibrate the methylene blue peak potential location. 3 sets of 3 electrodes are run to determine the standard calibration curve of our pH sensor.

Next, ATCC strains of Proteus mirabilis is grown in clean urine purchased from BiolVT in culture tubes at 37° C. where it will colonize and produce ureases. The urease will produce ammonia and raise the pH, which will then be measured with the sensors and compared to the bacterial load measured by a hemocytometer. The pH will also be verified by a pH probe. These measurements will help to determine the pH change based off CFU/mL infection level.

If the urine contain biofoulants that affect the monitoring capabilities, a filter membrane is integrated over the electrode. Immediate testing of sensors in urine samples does not indicate biofouling to be a significant issue. Electrochemical sensors tested in urine for an hour were not affected by biofouling. Also, the DNA tether may need to be adjusted if signal variation is observed.

### Example 5. Development of Electrochemical Sensor for Pseudomonas

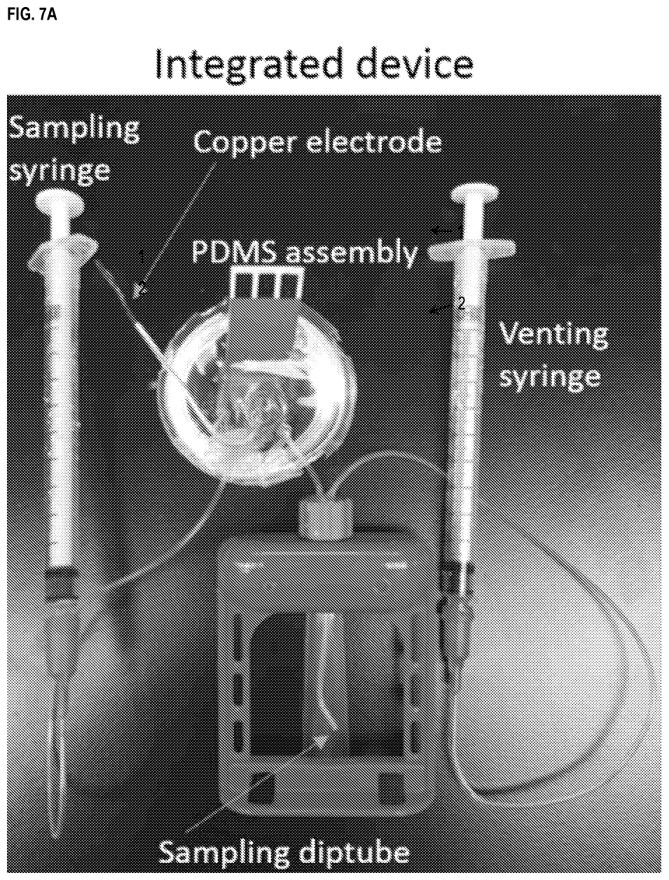

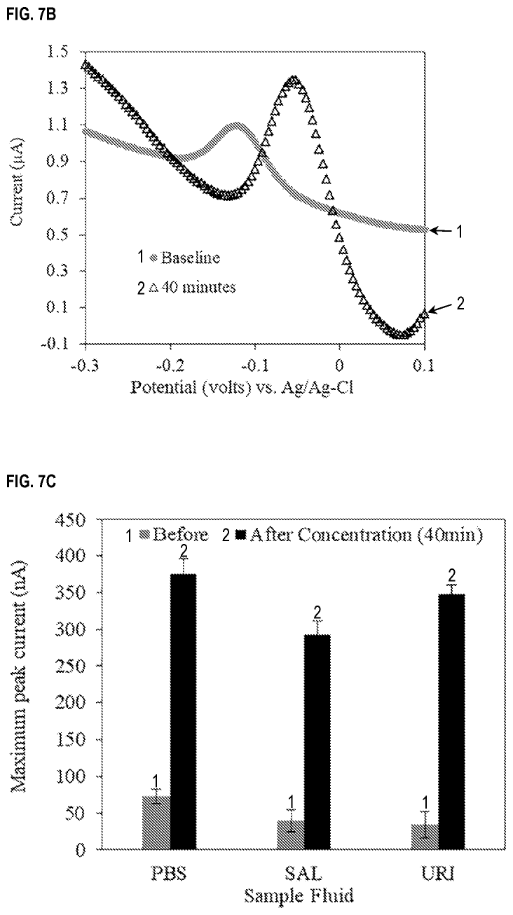

Pseudomonas aeruginosa was detected based on an electrochemical sensor for pyocyanin, using an osmotic system that was constructed as shown in FIG. 7A. Kimani et al., Biosample Concentration Using Microscale Forward Osmosis with Electrochemical Monitoring, Analytical Chemistry 91(11): 7487-7494 (2019). Briefly, commercially available dialysis grade cellulose ester membranes with 100-500 Da MWCO sizes, low density polyethene (LDPE) tubing with a 580 μm inner diameter and 960 μm outer diameter (Smiths Medical Part 800/100/200), polypropylene tubing connectors with 15 and 23 gauge stainless steel ends (Component supply part NE-231PL), Luer lock syringes with one milliliter volumes (VWR part BD-309628) were used. Dow Corning Sylgard 184 silicone elastomer base and curing agent (0.5 kg kit) were purchased from Ellsworth Adhesives (Germantown, WI) to make polydimethlsilosane (PDMS). Electrochemical measurements were carried out using disposable, screen-printed electrode sensors made by Zensor, and purchased from CH Instruments (part TE100), that consist of carbon working (3 mm diameter) and counter electrodes and a silver/silver-chloride (Ag/AgCl) reference. Copper electrodes were fabricated in-house.

A PDMS-enclosed microchannel for inline sample analysis was fabricated by making a mold using 7.5 mm glass slides and a packaging tape. A layer of the tape was cut into 9 mm long by 9 mm wide squares and placed onto the glass slide to create a microchannel covering the entire Zensor sensor area. Aluminum foil was used to surround the edges of the slide. A 30 milliliter volume of 10:1 ratio of PDMS base to curing agent was mixed and poured onto the mold ensuring that the final PDMS level was above the tape features. The assembly was then degassed under vacuum for 15 minutes and subsequently placed in an oven for curing at 80° C. for 1 hour. The cured PDMS was then removed from the master, and the edges were trimmed using a razor blade. Three access holes were drilled using 15 gauge stainless steel needles for inlet/outlet tubing connections and copper electrode placement in the microchannel. A hot glue gun was used to seal the copper electrode and tubing onto the PDMS assembly. Finally, the microchannel was super-glued onto the disposable carbon electrode. A 23 gauge needle with a Luer lock was inserted at the end of the outlet tubing, and a one milliliter syringe was connected to serve as the sampling device.

A hole was drilled through the membrane compartment cap used to seal the membrane compartment. The secondary tubing from the PDMS assembly was inserted into the membrane compartment to serve as a dip-tube for sample recovery from the membrane. An outlet tubing for venting gas was also inserted at the top of the membrane compartment. A 23 gauge needle with Luer lock was inserted at the other end of the venting tubing and a one milliliter syringe was connected to serve as the venting device. The dip-tube and venting tubing were superglued on the Luer lock to form a tight air seal.

Voltammetric electrochemical measurements were performed using a potentiostat from CH Instruments (part CHI 1040C). Impedance spectroscopy measurements were performed using an electrochemical workstation supplied by Zahner-Electrik (part I M6ex). Once the device was fabricated, it was tested with deionized water to ensure all seals were intact and to measure the total fluid volume in the tubing and compartments.

Stock solutions of 10 mM PBS were prepared to investigate the extent of sample concentration. In addition, urine and saliva samples from three healthy individuals were collected for testing of relevant biological samples. Urine and saliva contain complex compounds that can lead to membrane fouling that may further diminish sample concentration using the osmotic system. The osmotic system was then coupled with electrochemical detection to second derivatives of the faradaic peak response were used to calculate half of the peak height at each tested pyocyanin concentration. The output of this calculation is referred to as the maximum peak current. The three fluid samples (PBS, saliva, and urine) were each spiked with 1 μM pyocyanin. A 1 mL sample of each test fluid was then placed in the inside compartment of the osmotic system, while 5 M sucrose was placed as the draw solution to concentrate the samples for a duration of 40 min. Triplicate test samples were performed. Initial and final SWV scans were obtained to measure the pyocyanin redox peaks (FIG. 7B). All three test fluids were found to yield a 350-400% peak signal increase after 40 min of concentration, as shown in FIG. 7C, which corresponds to an up to 5-fold electrochemical signal increase. The fluid complexity did not have a considerable impact on the electrochemical signal amplification, as all three fluids showed similar results after being concentrated for 40 min.

These results demonstrate that the electrochemical sensors disclosed here are capable of detecting pyocyanin, and thereby detect Pseudomonas aeruginosa.

### Example 6. Printing of Four Electrochemical Sensors on a Disposable Printed Circuit Board

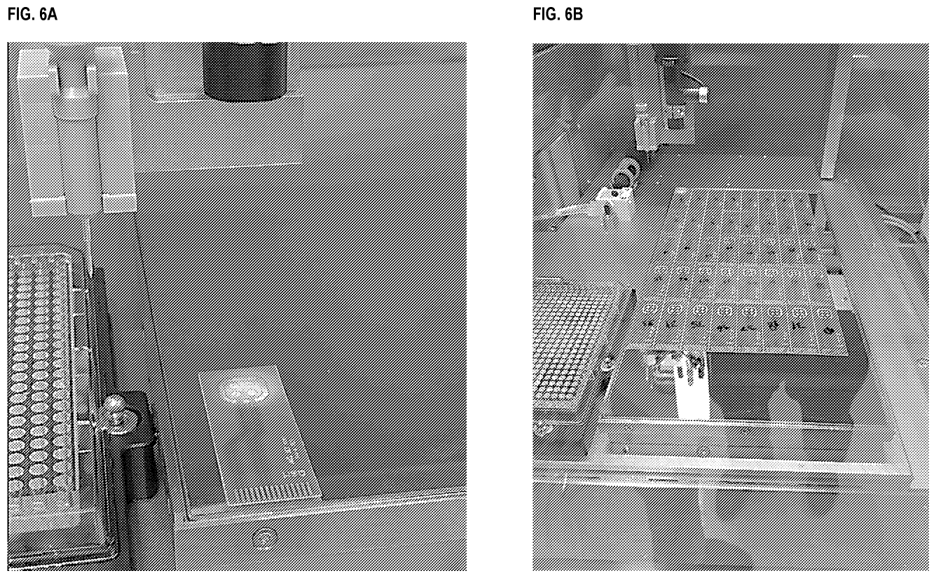

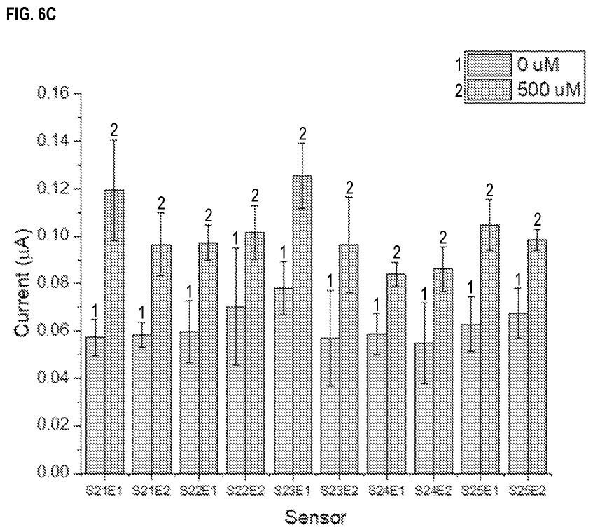

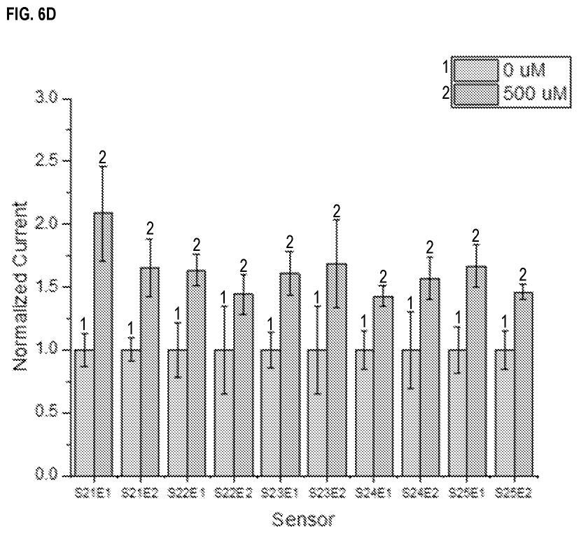

Protocols for hand functionalizing aptamers onto gold electrodes were adjusted to work with an M2 bioprinter for mass production of aptamer functionalized sensors. DNA was inkjet printed on disposable printed circuit boards and detected using UV light. As shown in FIG. 6A, printed DNA could be visualized on disposable printed circuit boards.

Printing was further tested and validated with kanamycin aptamer and target. Kanamycin aptamer was inkjet printed on disposable printed circuit boards. FIG. 6B shows mass production of disposable printed circuit boards printed with kanamycin aptamer onto gold electrodes. To test the kanamycin aptamer electrochemical sensor, first scans in a blank solution were performed to gain a baseline measurement prior to introducing a target containing solution to gain the testing measurement. Subsequently 500 μM kanamycin was used to analyze the function of the kanamycin aptamer electrochemical sensor. All measurements are in comparison to the baseline on each individual electrode. Raw data from these measurements are shown in FIG. 6C. As shown in FIG. 6C, each of the kanamycin aptamer electrochemical sensor showed an increased current in the presence of kanamycin compared to the current in absence of kanamycin. The raw data showed consistency in baseline measurements indicating reproducible printing. The response signal increased upon target binding and is statistically above the baseline levels. FIG. 6D shows the normalized data from the measurements shown in FIG. 6C.

These results indicate that inkjet printing on disposable printed circuit boards produced reproducible printing.

### Example 7. Integration of the Sensor Array and Testing with Clinical Samples

Once each electrochemical test is developed, the tests are combined onto a single sensor array. This array of gold electrodes are electrodeposited onto a single printed circuit board as shown in FIG. 4A. Each individual test will occupy one working electrode on the array (the four inner dots in FIG. 4A) and utilize the same Ag/AgCl reference electrode and counter electrode to complete the typical 3 electrode system for electrochemical testing. The electrodes will cover the test for the E. coli, STEC, and Proteus.

One potential limitation of disposable electrodes are that the reference electrode can degrade very quickly in solution. We have identified a company, Zimmer and Peacock (United Kingdom), that produces long lasting screen-printed Ag/AgCl reference electrodes using a unique paste mixture, which are incorporated into our design. Their reference electrodes can last in solution for up to 2 weeks, which is ideal for infection monitoring.

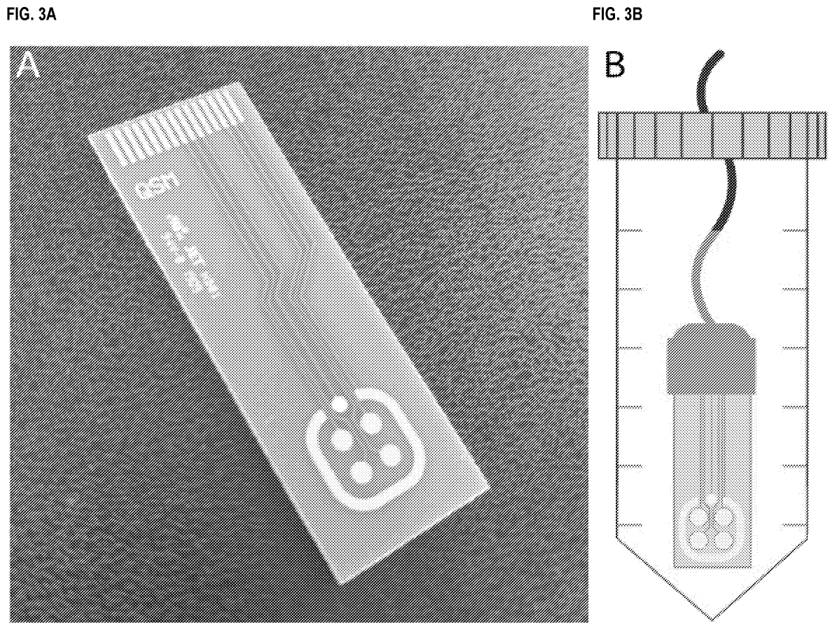

An off-the-shelf connector is available for the sensor shown in FIG. 3A. The sensor strip is permanently attached to the connector with a water-tight epoxy, and the wire exiting the connector is sealed with epoxy as well. The sensor, connector, and wire is placed inside of 50 mL Falcon tube. A hole is made in the bottle cap and the wire exiting the connector is pulled through the hole and the interface sealed as schematically shown in FIG. 3B. 100 prototype setups are fabricated using this method. The prototype setups are gamma sterilized by a third-party vendor prior to testing with urine samples.

The integrated sensor platform is tested with clean and spiked urine samples for two-week durations to validate the sensor stability. While the final product is not intended to be used for more than 1 week, sensor performance and variation needs to be characterized in case the product is used longer than intended. Our sensor array is capable of identifying and monitoring polymicrobial infections using the individual sensor combinations. Triplicates of the bacterial combinations, plus negative controls (clean urine), are tested with the sterile prototype setups. Once stability testing is completed, 15 known positive CAUTI urine samples are purchased from BiolVT and tested in a sterile container integrated with our sensor array for up to 1 week at room temperature.

### Example 8. The Electrochemical Sensor Array

To build an electrochemical sensor array, the three categories of electrochemical tests that are used to identify the presence of common bacteria are an aptamer-based metabolite binding test, a pH test, and an electroactive chemical species detection test. Individually, any one of these three tests does not successfully detect all of the common uropathogenic bacterial species. Table 1 organizes the sensors are created based on the pathogen and metabolite produced.

TABLE 1
Metabolites and electrochemical detection
method for pathogens found in CAUTI.

| | | Electrochemical Method |
|---|---|---|
| Pathogen | Metabolite Produced | of Detection |
| E. coli | Enterobactin | Aptamer-based test |
| STEC | Shiga toxin | Aptamer-based test |
| Proteus | Urease → ammonia | pH test |
| Pseudomonas | Pyocyanin | Electroactive species test |

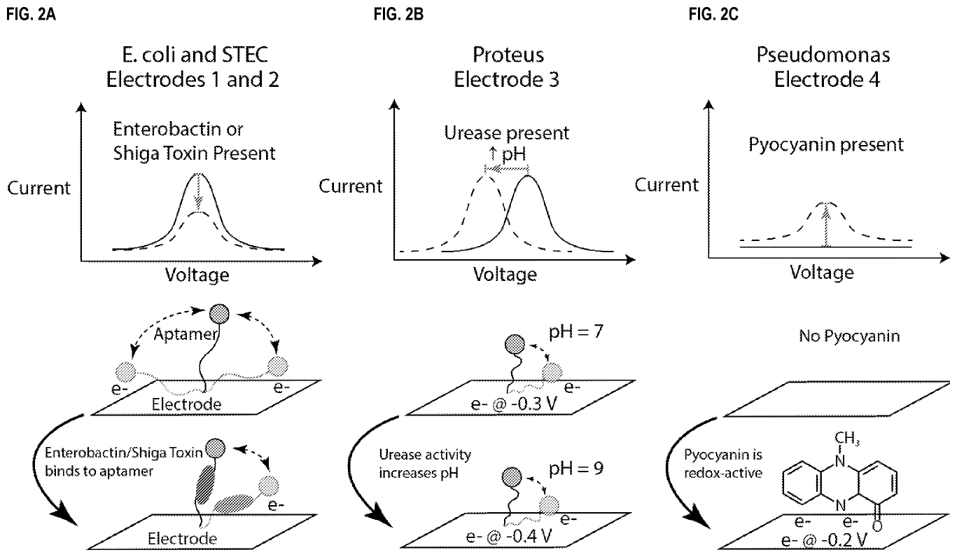

An aptamer-based electrochemical sensor is used to detect Escherichia coli based on two different metabolites, enterobactin and Shiga toxin. Continuous monitoring of metabolites in urine using aptamer-based sensors has not been demonstrated previously. In urinary tract infections, E. coli secrete large quantities of enterobactin to capture the limited iron available, which the bacteria require for their normal cellular function. There are also some strains of E. coli that produce Shiga toxin as a virulence factor. The infections from Shiga toxin-producing E. coli (STEC) are known to be more dangerous to patients. A panel of two different aptamers are used: one that binds to enterobactin, and one that binds to Shiga toxin to detect E. coli or the more serious STEC. We have previously developed aptamers that specifically bind to enterobactin and Shiga toxin. By modifying these aptamers with a redox probe and tethering them to an electrode surface, specific binding of the enterobactin or Shiga toxin to their corresponding aptamer will result in a conformational change of the aptamer that alters the peak current generated from the attached redox molecule. FIG. 2A shows an illustrative aptamer-based sensor, which works by measuring a peak current decrease upon enterobactin or Shiga toxin binding to the aptamer, due to the more constricted mobility of the aptamer and redox probe. Deviations from the baseline peak height indicate binding and thus the presence of these secreted molecules, indicative of an E. coli infection as seen schematically in FIG. 2A. Each aptamer is attached to their own electrode to distinguish between the individual metabolites. We have currently demonstrated one Shiga toxin aptamer-electrochemical sensor to detect down to 1 μM Shiga toxin concentrations in urine (physiologically relevant (See FIG. 5)). The enterobactin aptamer candidates are fully characterized and optimized.

Next, a pH test is used to detect Proteus mirabilis. Proteus secretes a large amount of ureases during a urinary tract infection, which convert urea into ammonia. The build-up of ammonia causes an increase in pH in the urine, which can be detected electrochemically by measuring the potential (voltage) difference of the solution. When combined with a stable reference electrode, the potential of certain redox molecules shifts in a predictable manner based on the pH. By monitoring the current peak potential location, it is possible to determine the pH of the urine. No other bacteria commonly found in CAUTI produces as much urease or any other enzyme that may act to significantly increase the pH of the urine, making this test a unique identifier of a Proteus infection. FIG. 2B shows an illustrative pH sensor, which works by measuring the voltage at which a peak current occurs for a pH-sensitive redox molecule. An exemplary pH-sensitive redox molecule is methylene blue. The peak current voltage is observed at more negative values with an increase in pH.

Lastly, an electroactive (redox) species detection test is used to detect a Pseudomonas aeruginosa infection. P. aeruginosa secretes a unique metabolite, pyocyanin, which is redox active. Pyocyanin can exchange electrons with an electrode at a given potential to produce a measurable current, as illustrated in FIG. 2C. No other bacterial species produces this molecule, or any molecule that results in a peak current at the same electrochemical potential as pyocyanin, making this test a unique identifier of a Pseudomonas aeruginosa infection. We are the leading experts in electrochemical detection of pyocyanin, and our results have shown correlations between pyocyanin levels and bacteria population (CFUs/mL). We have also shown that this electrochemical method works in urine and other biological fluids through testing over 100 clinical isolates.

These results demonstrate that an electrochemical sensor array that comprises multiple sensors may be assembled. Such arrays may be used for detection of multiple pathogens in biological samples, such as urine, stool and wound exudate.

### Example 9. The Electrochemical Detection of Pseudomonas Aeruginosa in Biological Samples

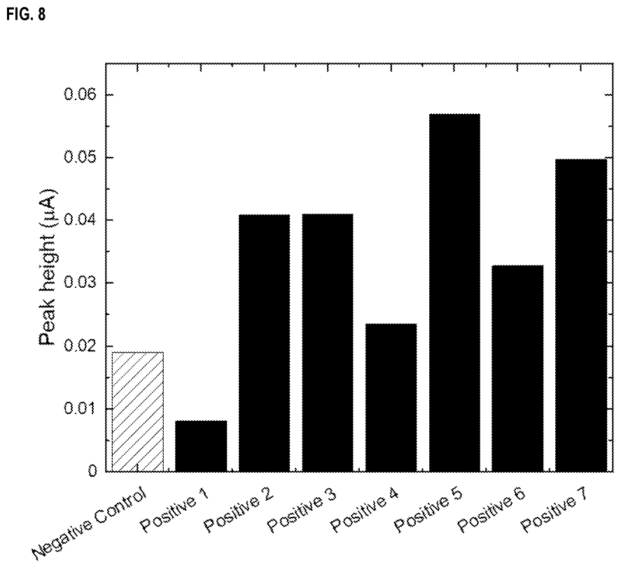

An electroactive (redox) species detection test was used to detect a Pseudomonas aeruginosa infection in human urine samples. The detection of Pseudomonas aeruginosa was carried out on the basis of pyocyanin, a unique metabolite, which is redox active. Samples were collected directly from patients with urinary tract infections. No sample preparation or alteration was performed prior to analysis. A culture negative urine sample was used as a negative control. The urine samples were directly applied to the electrochemical sensors for testing, and the peak current at the same electrochemical potential as pyocyanin was measured. Peak height was plotted for all samples. As shown in FIG. 8, six out of seven culture positive Pseudomonas aeruginosa urine samples were electrochemically detected using the electrochemical sensors with peaks higher than the background level of a culture negative urine control.

These results demonstrate that an electrochemical sensor array disclosed herein can detect a pathogen in a biological sample, such as urine.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present technology is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A method of detecting an infection in urine of a subject, the method comprising
(i) contacting urine from the subject with a device for detecting an infection, wherein the device comprises an electrochemical sensor array; and
(ii) measuring a presence, absence or amount of one or more target molecule and/or a metabolic activity within the urine sample, wherein the target molecule and/or the metabolic activity is associated with the infection, wherein the electrochemical sensor array performs the measuring.

2. A method of detecting a urinary tract infection in a subject, the method comprising
(i) contacting urine from the subject with a device for detecting an infection, wherein the device comprises an electrochemical sensor array; and
(ii) measuring a presence, absence or amount of one or more target molecule and/or a metabolic activity within the urine sample, wherein the target molecule and/or the metabolic activity is associated with the infection, wherein the electrochemical sensor array performs the measuring.

3. The method of claim 1 or claim 2, wherein the urine is collected in a catheter bag or a urine collection bag.

4. The method of any one of claims 1 to 3, wherein the urinary tract infection is catheter associated urinary tract infections (CAUTI).

5. A method of detecting a wound infection in a subject, the method comprising
(i) contacting wound exudate from the subject with a device for detecting an infection, wherein the device comprises an electrochemical sensor array; and
(ii) measuring a presence, absence or amount of one or more target molecule and/or a metabolic activity within the wound exudate, wherein the target molecule and/or the metabolic activity is associated with the infection, wherein the electrochemical sensor array performs the measuring.

6. The method of claim 5, wherein the wound exudate is collected in a wound dressing or a wound exudate collection container.

7. A method of detecting a wound infection in a subject, the method comprising:
(i) administering a dressing to a wound, optionally wherein the dressing comprises oxidized regenerated cellulose (ORC) and/or collagen,
(ii) applying a negative pressure to the wound,
(iii) collecting wound exudate in a wound exudate collection container,
(iv) contacting wound exudate from a wound dressing or a wound exudate collection container with a device for detecting an infection, wherein the device comprises an electrochemical sensor array; and
(v) measuring a presence, absence or amount of one or more target molecule and/or a metabolic activity within the wound exudate, wherein the target molecule and/or the metabolic activity is associated with the infection, wherein the electrochemical sensor array performs the measuring.

8. The method of any one of claims 1 to 7, further comprising estimating a number of viable cells of a pathogen associated with the infection based on the presence, absence or amount of the target molecule and/or the metabolic activity.

9. The method of any one of claims 1 to 8, wherein the method informs the withholding of one or more antibiotics upon a negative test for infection.

10. The method of any one of claims 1 to 9, wherein the method informs the selection of an appropriate antibiotic for the infection upon a positive test for infection.

11. The method of claim 10, further comprising administering an appropriate antibiotic for the infection upon a positive test for infection.

12. The method of any one of claims 1 to 11, wherein the electrochemical sensor array further comprises a sensor selected from a pH sensor and a temperature sensor.

13. The method of any one of claims 1 to 12, wherein the electrochemical sensor array detects a change in pH, a change in temperature, an electrochemical reaction, binding to an aptamer, a change in color, and the combination of any two or more thereof.

14. The method of any one of claims 1 to 13, wherein each electrochemical sensor of the electrochemical sensor array independently performs an electrochemical measurement.

15. The method of claim 14, wherein the electrochemical measurement is selected from square wave voltammetry, linear sweep voltammetry, staircase voltammetry, cyclic voltammetry, normal pulse voltammetry, differential pulse voltammetry, and chronoamperometry.

16. The method of claim 14 or claim 15, wherein the electrochemical measurement is square wave voltammetry and the current flow is measured in response to one or more square wave potentials.

17. The method of any one of claims 1 to 16, wherein each electrochemical sensor comprises a second working electrode, wherein the working electrode is one of an oxidizing electrode and a reducing electrode, and the second working electrode is the other of the oxidizing electrode and the reducing electrode.

18. The method of claim 17, wherein the working electrode is comprised of gold (Au), silver (Ag), platinum (Pt), indium tin oxide (ITO), carbon, carbon nanotubes, carbon nanofibers, graphene, carbon-platinum composites, carbon nanotubes with gold nanoparticles, and any combination thereof.

19. The method of claim 17 or claim 18, wherein the electrochemical sensor further comprise a reference electrode, optionally wherein the reference electrode is comprised of silver (Ag), silver chloride (AgCl), and platinum (Pt), and any combination thereof.

20. The method of any one of claims 1 to 19, wherein the infection is caused by a pathogen.

21. The method of claim 20, wherein the pathogen is selected from a bacterium and a fungus.

22. The method of claim 21, wherein the bacterium is selected from Pseudomonas aeruginosa, Proteus mirabilis, Escherichia coli, Klebsiella pneumoniae, other Klebsiella species, Staphylococcus aureus, Enterococcus faecalis, other Enterococcus species, Acinetobacter baumannii, Streptococcus group A species, Streptococcus Group B species, Staphylococcus epidermidis, Pseudomonas aeruginosa, Clostridium difficile, and Salmonella enterica.

23. The method of claim 21, wherein the fungus is selected from Candida albicans, Cryptococcus neoformans, and Aspergillus species.

24. The method of claim 23, wherein the fungus is Candida albicans.

25. The method of any one of claims 1 to 24, wherein the device is capable of contemporaneously detecting the presence or absence of at least two, or at least 3, or at least 4, or at least 5, or at least 6, or at least 7, or at least 8, or at least 9, or at least 10 pathogens.

26. The method of any one of claims 1 to 25, wherein the device is capable of contemporaneously detecting at least two, or at least three, or at least 4 signals.

27. The method of claim 26, wherein the signal is associated with the pathogen.

28. The method of any one of claims 1 to 27, wherein the device detects a target molecule, and/or a metabolic activity of the pathogen.

29. The method of claim 28, wherein the target molecule is a quorum sensing molecule.

30. The method of claim 28 or claim 29, wherein the target molecule is a redox molecule.

31. The method of claim 28, wherein the metabolic activity causes breakdown of a basic molecule.

32. The method of claim 31, wherein the metabolic activity is a urease activity.

33. The method of claim 31 or claim 32, wherein the metabolic activity changes pH of the urine or the wound exudate.

34. The method of claim 33, wherein the change in pH is an increase in pH.

35. The method of any one of claims 28 to 34, wherein the device detects the presence, absence or amount of the target molecule and/or the metabolic activity and/or the metabolic activity of the pathogen.

36. The method of claim 35, wherein the presence, absence or amount of the target molecule and/or the metabolic activity is measured as current flow through the working electrode.

37. The method of claim 35 or claim 36, wherein the presence, absence or amount of the target molecule and/or the metabolic activity is measured as current flow through the oxidizing electrode and the reducing electrode.

38. The method of claim 35 or claim 36, wherein the presence, absence or amount of the target molecule and/or the metabolic activity is measured as a change in pH.

39. The method of any one of claim 28 to 30 or 35-38, wherein the target molecule is selected from 3O-C12-homoserine lactone (3OXO), putrescine, Shiga toxin, aerobactin, auto inducing peptide-1 (AIP-1), gelatinase biosynthesis activating peptide (GBAP), EppR (Protein), short hydrophobic peptide 3 SHP3 (also known as SHP1520), autoinducing peptide (AIP), autoinducing peptide 2 (AIP 2), pyocyanin, enterobactin, tyrosol and farnesol.

40. The method of claim 39, wherein the presence of the target molecule is indicative of a presence and/or an amount of and/or a number of viable cells of the pathogen.

41. The method of claim 39 or claim 40, wherein the pathogen is selected from Pseudomonas aeruginosa, Proteus mirabilis, Escherichia coli, Klebsiella pneumoniae, other Klebsiella species, Staphylococcus aureus, Enterococcus faecalis, other Enterococcus species, Acinetobacter baumannii, Streptococcus group A species, Streptococcus Group B species, Staphylococcus epidermidis, Pseudomonas aeruginosa, Clostridium difficile, Salmonella enterica, Candida albicans, Cryptococcus neoformans, and Aspergillus species.

42. The method of claim 40, wherein the presence, absence or amount of:
3O-C12-homoserine lactone (3OXO) is indicative of the presence and/or the amount of and/or the number of viable cells of Pseudomonas aeruginosa,
putrescine is indicative of the presence and/or the amount of and/or the number of viable cells of Proteus mirabilis,
Shiga toxin is indicative of the presence and/or the amount of and/or the number of viable cells of Escherichia coli,
aerobactin is indicative of the presence and/or the amount of and/or the number of viable cells of Klebsiella and/or E. coli,
auto inducing peptide-1 (AIP-1) is indicative of the presence and/or the amount of and/or the number of viable cells of Staphylococcus aureus,
gelatinase biosynthesis activating peptide (GBAP) is indicative of the presence and/or the amount of and/or the number of viable cells of Enterococcus faecalis,
EppR protein is indicative of the presence and/or the amount of and/or the number of viable cells of Acinetobacter baumannii,
short hydrophobic peptide 3 SHP3 (SHP1520) is indicative of the presence and/or the amount of and/or the number of viable cells of Streptococcus agalactiae,
autoinducing peptide (AIR) is indicative of the presence and/or the amount of and/or the number of viable cells of Staphylococcus pseudintermedius,
autoinducing peptide 2 (AIP 2) is indicative of the presence and/or the amount of and/or the number of viable cells of Staphylococcus epidermidis,
pyocyanin is indicative of the presence and/or the amount of and/or the number of viable cells of Pseudomonas aeruginosa,
tyrosol is indicative of the presence and/or the amount of and/or the number of viable cells of Candida albicans,
farnesol is indicative of the presence and/or the amount of and/or the number of viable cells of Candida albicans, and/or
enterobactin is indicative of the presence and/or the amount of and/or the number of viable cells of uropathogenic Escherichia coll.

43. The method of any one of claims 1 to 42, wherein the device is electrically connected or connectable to a reader.

44. The method of claim 43, wherein the reader provides an output of a presence and/or an amount of and/or a number of viable cells of a pathogen.

45. The method of claim 43, wherein the pathogen is selected from a bacterium and a fungus.

46. The method of claim 44, wherein the pathogen is selected from Pseudomonas aeruginosa, Proteus mirabilis, Escherichia coli, Klebsiella pneumoniae, Staphylococcus aureus, Enterococcus faecalis, Acinetobacter baumannii, Streptococcus Group A species, Streptococcus Group B species, Staphylococcus epidermidis, Pseudomonas aeruginosa, Candida albicans, Cryptococcus neoformans, and Aspergillus species.

47. The method of claim 45 or 46, wherein the device detects the presence and/or the amount of and/or the number of viable cells of a pathogen in less than one hour, less than 45 minutes, or less than 30 minutes, or less than 15 minutes, or less than 10 minutes, or less than 5 minutes, or less than 2 minutes or less than 1 minute.

48. The method of any one of claims 1 to 44, wherein the device detects the infection in less than one hour, less than 45 minutes, or less than 30 minutes, or less than 15 minutes, or less than 10 minutes, or less than 5 minutes, or less than 2 minutes or less than 1 minute.


SEQUENCE LISTING (10 sequences)

SEQ ID NO: 1 - DNA, 80 bp, Artificial Sequence
  Synthetic Polypeptide
acccctgcatcctttgctggggtaactagcattcatttcccacacccgtcccgtccatatagtctagagggccccagaat

SEQ ID NO: 2 - DNA, 80 bp, Artificial Sequence
  Synthetic Sequence
attctggggccctctagactatatggacgggacgggtgtgggaaatgaatgctagttaccccagcaaaggatgcaggggt

SEQ ID NO: 3 - DNA, 39 bp, Artificial Sequence
  Synthetic Sequence
attagctatcttccacgattcgatcaggcagtacgtcgt

SEQ ID NO: 4 - DNA, 39 bp, Artificial Sequence
  Synthetic Sequence
acagttatccgactgctattcgatcaggcagtacgtagc

SEQ ID NO: 5 - DNA, 39 bp, Artificial Sequence
  Synthetic Sequence
caggctgttctgacgcataaggaatgcgctgttgcagag

SEQ ID NO: 6 - DNA, 39 bp, Artificial Sequence
  Synthetic Sequence
ttggtcctgctttggatagtcgcgaaaggggtgccactg

SEQ ID NO: 7 - DNA, 39 bp, Artificial Sequence; identical to SEQ ID NO: 4
  Synthetic Sequence

SEQ ID NO: 8 - DNA, 39 bp, Artificial Sequence
  Synthetic Sequence
accgagcggttttacgtctcaagtagtatcccgttttgc

SEQ ID NO: 9 - DNA, 39 bp, Artificial Sequence; identical to SEQ ID NO: 3
  Synthetic Sequence

SEQ ID NO: 10 - DNA, 56 bp, Artificial Sequence
  Synthetic Sequence
ttgccatcctgtactatgctctatcgggcggtttagtgatccttcgtccaactatc